(19) United States
(12) Patent Application Publication
(10) Pub. No.: US 2024/0317743 A1
(43) Pub. Date: Sep. 26, 2024

(54) Solid Forms of BCL-2 Inhibitors, Method of Preparation, and Use Thereof

(71) Applicants: BeiGene, Ltd., Camana Bay (KY); BeiGene Switzerland GmbH, Basel (CH)

(72) Inventors: Desheng Yu, Beijing (CN); Gongyin Shi, Beijing (CN); Hai Xue, Beijing (CN); Yunhang Guo, Beijing (CN)

(73) Assignees: BeiGene, Ltd., Camana Bay (KY); BeiGene Switzerland GmbH, Basel (CH)

(21) Appl. No.: 18/589,022

(22) Filed: Feb. 27, 2024

Related U.S. Application Data
(63) Continuation of application No. PCT/CN2022/116084, filed on Aug. 31, 2022.

(30) Foreign Application Priority Data
Aug. 31, 2021 (WO) PCT/CN2021/115718

(51) Int. Cl. C07D 471/04; A61K 31/438

(52) U.S. Cl. CPC C07D 471/04; A61K 31/438

(57) ABSTRACT

The present invention relates to a solid form, particularly a crystalline forms of Bcl-2 inhibitor 2-((1H-pyrrolo[2,3-b]pyridin-5-yl)oxy)-N-((4-((((1r,4r)-4-hydroxy-4-methylcyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)benzamide, pharmaceutical compositions comprising the solid form, processes for preparing the solid form, and methods of use therefore.

### CROSS-REFERENCE TO RELATED APPLICATIONS

This application is a continuation of International Application No. PCT/CN2022/116084, filed on Aug. 31, 2022, which claims priority to International Application No. PCT/CN2021/115718, filed on Aug. 31, 2021, the disclosures of each of which are hereby incorporated by reference in their entireties.

### FIELD OF THE DISCLOSURE

Disclosed herein are solid forms of Bcl-2 inhibitor 2-((1H-pyrrolo[2,3-b]pyridin-5-yl)oxy)-N-((4-((((1r,4r)-4-hydroxy-4-methylcyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)benzamide, pharmaceutical compositions comprising the solid form, processes for preparing the solid form, and methods of use therefore.

### BACKGROUND OF DISCLOSURE

Programmed cell death or apoptosis occurs in multicellular organisms to dispose damaged or unwanted cells, which is critical for normal tissue homeostasis. (Br. J. Cancer 1972, 26, 239). However defective apoptotic processes have been implicated in a wide variety of diseases. Excessive apoptosis causes atrophy, whereas an insufficient amount results in uncontrolled cell proliferation, such as cancer (Cell 2011, 144, 646). Resistance to apoptotic cell death is a hallmark of cancer and contributes to chemoresistance (Nat Med. 2004, 10, 789-799). Several key pathways controlling apoptosis are commonly altered in cancer. Some factors like Fas receptors and caspases promote apoptosis, while some members of the B-cell lymphoma 2 (Bcl-2) family of proteins inhibit apoptosis. Negative regulation of apoptosis inhibits cell death signaling pathways, helping tumors to evade cell death and developing drug resistance.

There are two distinct apoptosis pathways including the extrinsic pathway and the intrinsic pathway. The extrinsic pathway is activated in response to the binding of death-inducing ligands to cell-surface death receptors (Nat Rev Drug Discov. 2017 16, 273-284). The B cell lymphoma 2 (BCL-2) gene family, a group of proteins homologous to the Bcl-2 protein, encodes more than 20 proteins that regulate the intrinsic apoptosis pathway. Bcl-2 family proteins are characterized by containing at least one of four conserved Bcl-2 homology (BH) domains (BH1, BH2, BH3 and BH4) (Nat. Rev. Cancer 2008, 8, 121; Mol. Cell 2010, 37, 299; Nat. Rev. Mol. Cell Biol. 2014, 15, 49). Bcl-2 family proteins, consisting of pro-apoptotic and anti-apoptotic molecules, can be classified into the following three subfamilies according to sequence homology within four BH domains: (1) a subfamily shares sequence homology within all four BH domains, such as Bcl-2, Bcl-XL and Bcl-w which are anti-apoptotic; (2) a subfamily shares sequence homology within BH1, BH2 and BH4, such as Bax and Bak which are pro-apoptotic; (3) a subfamily shares sequence homology only within BH3, such as Bik, Bid and HRK which are pro-apoptotic. One of the unique features of Bcl-2 family proteins is heterodimerization between anti-apoptotic and pro-apoptotic proteins, which is considered to inhibit the biological activity of their partners. This heterodimerization is mediated by the insertion of a BH3 region of a pro-apoptotic protein into a hydrophobic cleft composed of BH1, BH2 and BH3 from an anti-apoptotic protein. In addition to the BH1 and BH2, the BH4 domain is required for anti-apoptotic activity. In contrast, BH3 domain is essential and, itself, sufficient for pro-apoptotic activity.

Similar to oncogene addiction, in which tumor cells rely on a single dominant gene for survival, tumor cells may also become dependent on Bcl-2 in order to survive. Bcl-2 overexpress is found frequently in acute myeloid leukemia (AML), acute lymphocytic leukemia (ALL), relapsed/refractory chronic lymphocytic leukemia (CLL), follicular lymphoma (FL), non-Hodgkin lymphoma (NHL) and solid tumors such as pancreatic, prostate, breast, and small cell and non-small cell lung cancers (Cancer 2001, 92, 1122-1129; Cancer Biol. 2003; 13:115-23; Curr. Cancer Drug Targets 2008, 8, 207-222; Cancers 2011, 3, 1527-1549). Dysregulated apoptotic pathways have also been implicated in the pathology of other significant diseases such as neurodegenerative conditions (up-regulated apoptosis), e.g., Alzheimer's disease; and proliferative diseases (down-regulated apoptosis), e.g., cancers, autoimmune diseases and pro-thrombotic conditions.

International publication WO2019/210828 disclosed a series of Bcl-2 inhibitors, in particularly, 2-((1H-pyrrolo[2,3-b]pyridin-5-yl)oxy)-N-((4-((((1r,4r)-4-hydroxy-4-methylcyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)benzamide (hereinafter Compound 1), which selectively inhibit Bcl-2 proteins for the treatment of dysregulated apoptotic diseases such as cancers, autoimmune diseases and pro-thrombotic conditions.

Compound 1 has 13 freely rotatable bonds and a high molecular weight (Mw>800). Molecules with a large degree of conformational flexibility tend to be extremely difficult to crystallize, and the most important molecular descriptors responsible for the crystallization behavior of these molecules were related to the number of rotatable bonds and the length of the alkyl side chains (Bruno C. Hancock. Predicting the Crystallization Propensity of Drug-Like Molecules. Journal of Pharmaceutical Sciences, 2017, 106: 28-30). In practice, for a particular compound especially with a large molecular weight and many freely rotatable bonds, it is not possible to predict that whether a pure physical form can be obtained, and which physical forms will be stable and suitable for pharmaceutical use. Similarly, it is equally impossible to predict whether a particular crystalline solid-state form can be produced with the desired chemical and physical properties suitable for pharmaceutical formulations.

For all the foregoing reasons, there is a great need to find crystalline forms of Compound 1 that provide good stability and good manufacturability. The present disclosure advantageously meets one or more of these requirements.

### SUMMARY OF THE DISCLOSURE

The present disclosure addresses the foregoing challenges and needs by providing solid from, preferably a crystalline form of Compound 1, which is suitable for pharmaceutical use. Although Compound 1 was found to have multiple freely rotatable bonds and a high molecular weight of more than 800), the inventor of the present disclosure unexpectedly found twenty-one crystalline forms for Compound 1, including six anhydrates (Forms B, S, U, M, F and N), four hydrates/anhydrates (Forms H, R, L and T), and eleven solvates (Forms A, C, D, E, G, I, J, K, O, P and Q), wherein isomorphism occurred during the formation of Form I, Form L is a metastable form, Form N and Form T can convert into each other during storage, and Form S was obtained by heating Form R to 150° C.

The inventors discovered that Form A was an EtOAc solvate of Compound 1, which possesses good physical properties, including better physical stability and better solubility. However, it is difficult to control the content of ethyl acetate of Form A during the manufacture, storage and formulation, and Form A can be converted into Form B after Form A is heated to 160° C., cooled back to room temperature and re-exposed to the air atmosphere.

Solvates Forms C, D, J, K and O and anhydrate Form F can be converted to anhydrate Form B after being heated to high temperatures; Forms K and F can spontaneously convert to Form B after long-time storage, and Form R can be converted to anhydrate Form S after being heated to 150° C.

Anhydrate Forms B, S, and M show better physicochemical stability compared with Forms F, H, N and R, when exposed under 25° C./60% RH and 40° C./75% RH for 1 week, and, 80° C./sealed for 24 hrs.

Furtherly, Form B has good thermodynamic stability with a high melting point and a slight hygroscopicity with 0.9% water uptake at 25° C./80% RH. It also showed good physicochemical and thermodynamic stability, after exposing under 25° C./80% RH, and shaking in acetone/H₂O (1:9, v/v) and H₂O for about 4 days.

The inventors tried to scaled-up Form B, however, failed to obtain the desired form by routine crystallization methods directly, and had to heat Form A or treat Form K in certain solvents under a temperature about 100° C. to obtain Form B, which could not meet the requirements of the scaled-up process. Form M with good stability was obtained from the solvent of CHCl₃ and heptane as anhydrate form, but CHCl₃ is not friendly with the environment and belongs to Class 2 with low 0.6 mg/day of permitted daily exposure (PDE) from ICH guideline. Form U as an anhydrate of Compound 1 was unexpectedly obtained by replacing CHCl₃ with DCM in the recrystallization step, and it is also reproducible and suitable for the scaled-up process. Form U showed good physicochemical, thermodynamic and physical stability, such as no significant chemical purity change, no crystal form, and no optical purity changes occurred when stored at 25±2° C./60±5% RH, or 40±2° C./75±5% RH conditions for up to 6 months. In addition, only Form U can remove a key dimer impurity in manufacture, which is a process impurity formed by the reaction between an acid intermediate (S)-2-((1H-pyrrolo[2,3-b]pyridin-5-yl)oxy)-4-(2-(2-(2-isopropylphenyl)pyrrolidin-1-yl)-7-azaspiro[3.5]nonan-7-yl)benzoic acid with Compound 1, effectively.

Although Form U has a lower melting point than Form B, Form U has no challenges from such as the issues of preparation, scaled-up process, solvent residue, and qualification of API and pharmaceutical formulations, and has good stability and the capability of formation via solution crystallization. Therefore, Form U is more suitable for manufacture and pharmaceutical formulations.

In a first aspect, disclosed herein is a crystalline form of 2-((1H-pyrrolo[2,3-b]pyridin-5-yl)oxy)-N-((4-((((1r,4r)-4-hydroxy-4-methylcyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)benzamide, designated as Form A.

In a second aspect, disclosed herein is a crystalline form of Compound 1, which is an EtOAc solvate, containing about 1 mol of EtOAc per mol.

In some embodiments, the crystalline form has an X-ray powder diffraction pattern comprising diffraction peaks having ° 2θ angle values at 16.5±0.1° and 24.5±0.1°.

In some embodiments, the crystalline form has an X-ray powder diffraction pattern comprising diffraction peaks having ° 2θ angle values at 12.4±0.1°, 16.5±0.1° and 24.5±0.1°.

In some embodiments, the crystalline form has an X-ray powder diffraction pattern comprising diffraction peaks having ° 2θ angle values at 12.4±0.1°, 16.5±0.1°, 20.7±0.1° and 24.5±0.1°.

In some embodiments, the crystalline form has an X-ray powder diffraction pattern comprising diffraction peaks having ° 2θ angle values at 10.6±0.1°, 12.4±0.1°, 16.5±0.1°, 20.7±0.1° and 24.5±0.1°.

In some embodiments, the crystalline form has an X-ray powder diffraction pattern comprising diffraction peaks having ° 2θ angle values at 10.6±0.1°, 12.4±0.1°, 13.8±0.1°, 16.5±0.1°, 20.7±0.1° and 24.5±0.1°.

In some embodiments, the crystalline form has an X-ray powder diffraction pattern comprising diffraction peaks having ° 2θ angle values at 10.6±0.1°, 12.4±0.1°, 13.8±0.1°, 14.1±0.1°, 16.5±0.1°, 20.7±0.1° and 24.5±0.1°.

In some embodiments, the crystalline form has an X-ray powder diffraction pattern comprising diffraction peaks having ° 2θ angle values at 10.6±0.1°, 12.4±0.1°, 13.8±0.1°, 14.1±0.1°, 16.5±0.1°, 17.0±0.1°, 20.7±0.1° and 24.5±0.1°.

In some embodiments, the crystalline form has an X-ray powder diffraction pattern comprising diffraction peaks having ° 2θ angle values at 10.6±0.1°, 12.4±0.1°, 13.8±0.1°, 14.1±0.1°, 16.5±0.1°, 17.0±0.1°, 19.5±0.1°, 20.7±0.1° and 24.5±0.1°.

In some embodiments, the crystalline form has an X-ray powder diffraction pattern comprising diffraction peaks having ° 2θ angle values at 6.9±0.1°, 10.6±0.1°, 12.4±0.1°, 13.8±0.1°, 14.1±0.1°, 16.5±0.1°, 17.0±0.1°, 19.5±0.1°, 20.7±0.1° and 24.5±0.1°.

In some embodiments, the crystalline form has an X-ray powder diffraction pattern comprising diffraction peaks having ° 2θ angle values at 6.9±0.1°, 7.4±0.1°, 8.8±0.1°, 10.6±0.1°, 10.9±0.1°, 12.4±0.1°, 12.7±0.10, 13.1±0.10, 13.4±0.1°, 13.8±0.10, 14.1±0.10, 14.7±0.10, 14.9±0.1°, 15.4±0.1°, 16.2±0.10, 16.5±0.1°, 17.0±0.1°, 17.5±0.1°, 18.2±0.1°, 18.5±0.1°, 19.1±0.1°, 19.5±0.1°, 20.7±0.1°, 21.1±0.1°, 21.8±0.1°, 22.4±0.1°, 22.8±0.1°, 23.3±0.1°, 23.8±0.1°, 24.1±0.1°, 24.5±0.1°, 25.8±0.1°, 26.7±0.1°, 27.1±0.1°, 27.6±0.1°, and 29.8±0.1°.

In some embodiments, Form A has an XRPD pattern substantially as shown in FIG. 1A or FIG. 1E.

In some embodiments, Form A is characterized by having two endotherm peaks at about 150° C. and about 178° C. by differential scanning calorimetry (DSC).

In some embodiments, Form A has a DSC thermogram substantially as shown in FIG. 1B.

In some embodiments, Form A is characterized by a crystal system of triclinic and the space group is P1 having the cell parameters: (a) is about 13.644 Å, (b) is about 14.070 Å, (c) is about 15.012 Å, (α) is about 112.0202(3) °, (μ) is about 104.6821(3) °, and (γ) is about 93.6507(2) °.

In a second aspect, disclosed herein is a crystalline form of Compound 1 is an anhydrate designated as Form B.

In some embodiments, the crystalline form has an X-ray powder diffraction pattern comprising diffraction peaks having ° 2θ angle values at 14.4±0.1°.

In some embodiments, the crystalline form has an X-ray powder diffraction pattern comprising diffraction peaks having ° 2θ angle values at 14.4±0.1° and 17.5±0.1°.

In some embodiments, the crystalline form has an X-ray powder diffraction pattern comprising diffraction peaks having ° 2θ angle values at 14.4±0.1°, 17.5±0.1° and 18.4±0.1°.

In some embodiments, the crystalline form has an X-ray powder diffraction pattern comprising diffraction peaks having ° 2θ angle values at 14.4±0.1°, 17.5±0.1°, 18.4±0.1° and 19.6±0.1°.

In some embodiments, the crystalline form has an X-ray powder diffraction pattern comprising diffraction peaks having ° 2θ angle values at 7.2±0.1°, 14.4±0.1°, 17.5±0.1°, 18.4±0.1° and 19.6±0.1°.

In some embodiments, the crystalline form has an X-ray powder diffraction pattern comprising diffraction peaks having 020 angle values at 6.7±0.1°, 7.2±0.1°, 13.8±0.1°, 14.4±0.1°, 17.5±0.1°, 18.4±0.1° and 19.6±0.1°.

In some embodiments, the crystalline form has an X-ray powder diffraction pattern comprising diffraction peaks having ° 2θ angle values at 6.7±0.1°, 7.2±0.1°, 13.8±0.1°, 14.4±0.1°, 17.5±0.1°, 18.4±0.1° and 19.6±0.1°.

In some embodiments, the crystalline form has an X-ray powder diffraction pattern comprising diffraction peaks having ° 2θ angle values at 6.7±0.1°, 7.2±0.1°, 11.6±0.1°, 12.2±0.1°, 13.3±0.1°, 13.8±0.1°, 14.4±0.1°, 15.7±0.1°, 16.2±0.1°, 17.5±0.1°, 18.4±0.1°, 19.6±0.1°, 19.9±0.1°, 23.0±0.1° and 24.9±0.1°.

In some embodiments, has an XRPD pattern substantially as shown in FIG. 2A or FIG. 2D.

In some embodiments, Form B is characterized by having one endotherm peak at about 187° C. by differential scanning calorimetry (DSC).

In some embodiments, Form B has a DSC thermogram substantially as shown in FIG. 2B.

In a third aspect, disclosed herein is a crystalline form of Compound 1 is an anhydrate designated as Form U.

In some embodiments, the crystalline form has an X-ray powder diffraction pattern comprising diffraction peaks having ° 2θ angle values at 11.3±0.1° and 24.3±0.1°.

In some embodiments, the crystalline form has an X-ray powder diffraction pattern comprising diffraction peaks having ° 2θ angle values at 11.3±0.1°, 15.6±0.1° and 24.3±0.1°.

In some embodiments, the crystalline form has an X-ray powder diffraction pattern comprising diffraction peaks having ° 2θ angle values at 11.3±0.1°, 15.6±0.1°, 21.2±0.1° and 24.3±0.1°.

In some embodiments, the crystalline form has an X-ray powder diffraction pattern comprising diffraction peaks having ° 2θ angle values at 11.3±0.1°, 13.5±0.1°, 15.6±0.1°, 21.2±0.1° and 24.3±0.1°.

In some embodiments, the crystalline form has an X-ray powder diffraction pattern comprising diffraction peaks having ° 2θ angle values at 11.3±0.1°, 13.5±0.1°, 15.6±0.1°, 17.0±0.1°, 21.2±0.1° and 24.3±0.1°.

In some embodiments, the crystalline form has an X-ray powder diffraction pattern comprising diffraction peaks having ° 2θ angle values at 11.3±0.1°, 13.5±0.1°, 15.6±0.1°, 17.0±0.1°, 19.5±0.1°, 21.2±0.1° and 24.3±0.1°.

In some embodiments, the crystalline form has an X-ray powder diffraction pattern comprising diffraction peaks having ° 2θ angle values at 7.0±0.1°, 11.3±0.1°, 13.5±0.1°, 15.6±0.1°, 17.0±0.1°, 19.5±0.1°, 21.2±0.1° and 24.3±0.1°.

In some embodiments, the crystalline form has an X-ray powder diffraction pattern comprising diffraction peaks having ° 2θ angle values at 7.0±0.1°, 11.3±0.1°, 13.5±0.1°, 15.6±0.1°, 17.0±0.1°, 19.5±0.1°, 20.0±0.1°, 21.2±0.1° and 24.3±0.1°.

In some embodiments, the crystalline form has an X-ray powder diffraction pattern comprising diffraction peaks having ° 2θ angle values at 7.0±0.1°, 9.4±0.1, 11.3±0.1°, 13.5±0.1°, 15.6±0.1°, 17.0±0.1°, 19.5±0.1°, 20.0±0.1°, 21.2±0.1° and 24.3±0.1°.

In some embodiments, the crystalline form has an X-ray powder diffraction pattern comprising diffraction peaks having ° 2θ angle values at 7.0±0.1°, 9.4±0.1, 11.3±0.1°, 13.5±0.1°, 15.6±0.1°, 17.0±0.1°, 17.5±0.1°, 19.5±0.1°, 20.0±0.1°, 21.2±0.1° and 24.3±0.1°.

In some embodiments, the crystalline form has an X-ray powder diffraction pattern comprising diffraction peaks having ° 2θ angle values at 7.0±0.1°, 9.4±0.1, 11.3±0.1°, 13.5±0.1°, 15.6±0.1°, 16.1±0.1°, 17.0±0.1°, 17.5±0.1°, 19.5±0.1°, 20.0±0.1°, 21.2±0.1°, 21.6±0.1° and 24.3±0.1°.

In some embodiments, the crystalline form has an X-ray powder diffraction pattern comprising diffraction peaks having ° 2θ angle values at 7.0±0.1°, 9.4±0.1°, 10.2±0.1°, 10.7±0.1°, 11.3±0.1°, 13.5±0.1°, 13.9±0.1°, 14.9±0.1°, 15.0±0.1°, 15.6±0.1°, 16.1±0.1°, 17.0±0.1°, 17.1±0.1°, 17.5±0.1°, 18.0±0.1°, 18.4±0.1°, 18.9±0.1°, 19.2±0.1°, 19.5±0.1°, 20.0±0.1°, 20.5±0.1°, 21.2±0.1°, 21.6±0.1°, 22.3±0.1°, 22.6±0.1°, 22.9±0.1°, 23.6±0.1°, 24.3±0.1°, 25.7±0.1°, 25.8±0.1°, 26.1±0.1°, 27.6±0.1°, 28.5±0.1°, 28.9±0.1°, and 29.3±0.1°.

In some embodiments, Form U has an XRPD pattern substantially as shown in FIG. 21A.

In some embodiments, Form U is characterized by having one endotherm peak at about 164° C. by differential scanning calorimetry (DSC).

In some embodiments, Form U has a DSC thermogram substantially as shown in FIG. 21B.

In a fourth aspect, a crystalline form of Compound 1 is designated as Form C, Form D, Form E, Form F, Form G, Form H, Form I, Form J, Form K, Form L, Form M, Form N, Form O, Form P, Form Q, Form R, Form S or Form T.

In some embodiments, Form C, Form D, Form E, Form F, Form G, Form H, Form I, Form J, Form K, Form L, Form M, Form N, Form O, Form P, Form Q, Form R, Form S and Form T have an XRPD pattern substantially as shown in FIG. 3A, FIG. 4A, FIG. 5A, FIG. 6A, FIG. 7A, FIG. 8A, FIG. 9A, FIG. 10A, FIG. 11A, FIG. 12A, FIG. 13A, 14A, FIG. 15A, FIG. 16A, FIG. 17, FIG. 18A, FIG. 19A and FIG. 20, separately.

In some embodiments of all above aspects, the crystalline forms are at least 40%, 50%, 60%, 70%, 80%, 90% or 95% crystalline.

In a fifth aspect, disclosed herein is an amorphous form of 2-((1H-pyrrolo[2,3-b]pyridin-5-yl)oxy)-N-((4-((((1r,4r)-4-hydroxy-4-methylcyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)benzamide (Compound 1).

In some embodiments, the amorphous of Compound 1 has an XRPD pattern substantially as shown in FIG. 22A.

In some embodiments, the amorphous of Compound 1 is characterized by having a glass transition signal at about 127° C. (middle).

In some embodiments of, the amorphous of Compound 1 contains no more than 1%, 2%, 3%, 4%, 5% or 10% of a crystalline form of Compound 1.

In a sixth aspect, disclosed herein is a pharmaceutical composition comprising (a) a therapeutically effective amount of a solid form of Compound 1, preferably a crystalline form of Compound 1 disclosed herein or an amorphous form of Compound 1, and; (b) one or more pharmaceutically acceptable excipients.

In some embodiments, the crystalline form of Compound 1 is a crystalline form of an EtOAc solvate of Compound 1 containing about 1 mol of EtOAc per mol, and an anhydrate of Compound 1.

In some embodiments, the crystalline form of Compound 1 is Form A, Form B or Form U of Compound 1.

In some embodiments, the crystalline form of Compound 1 is Form C, Form D, Form E, Form F, Form G, Form H, Form I, Form J, Form K, Form L, Form M, Form N, Form O, Form P, Form Q, Form R, Form S or Form T of Compound 1.

In a seventh aspect, disclosed herein is a process for preparing a pharmaceutical solution of Compound 1, comprising dissolving a solid form of Compound 1, preferably a crystalline form of Compound 1 of claim 1 in a pharmaceutically acceptable solvent or a mixture of solvents, or an amorphous form of Compound 1.

In eights aspect, disclosed herein is a method of treating a disease related to Bcl-2 proteins inhibition, comprising administering to a subject a therapeutically effective amount of a crystalline form of Compound 1, an amorphous form of Compound 1, or a pharmaceutical composition disclosed herein.

In some embodiments, the disease related to Bcl-2 proteins inhibition is a dysregulated apoptotic disease. In some preferred embodiments, the disease related to Bcl-2 proteins inhibition is a neoplastic, pro-thrombotic, immune or autoimmune disease.

In some embodiments, the crystalline form of Compound 1 is Form A, Form B or Form U of Compound 1.

In some embodiments, the crystalline form of Compound 1 is a crystalline form of an EtOAc solvate of Compound 1 containing about 1 mol of EtOAc per mol, or an anhydrate of Compound 1.

In some embodiments, the crystalline form of Compound 1 is Form C, Form D, Form E, Form F, Form G, Form H, Form I, Form J, Form K, Form L, Form M, Form N, Form O, Form P, Form Q, Form R, Form S or Form T of Compound 1.

In some embodiments, the therapeutically effective amount is orally administered at a dose of about 1 mg to about 640 mg Compound 1 per day.

In some embodiments, the subject is a human.

In some embodiments, Form A is obtained by the process comprising any one of the following procedures:
a) dissolving Compound 1 in DCM, removing DCM, charging with EA, to obtain Form A;
b) dissolving Compound 1 in DCM, concentrating, charging with EA, exchanging DCM with EA, MeOH and EA separately, to obtain Form A;
c) dissolving Compound 1 in EA, heating and cooling, to obtain Form A; or,
d) dissolving Compound 1 in THF/EtOAc (1:2, v/v) solvent mixture, evaporating, to obtain Form A.

In some embodiments, Form B is obtained by the process comprising any one of the following procedures:
a) dissolving Compound 1 in acetone, evaporating the solvent, to obtain the desired crystalline form;
b) heating Form A, Form C, Form O to about 160° C. and cooling back to RT, to obtain Form B;
c) heating Form A stepwise isothermally to about 100° C., to obtain Form B;
d) heating Form D or Form J to about 130° C. and being isothermal, to obtain Form B; or,
e) adding Form K into heptane, refluxing at about 100° C. and cooling, to obtain Form B.

In some embodiments, Form U is obtained by the process comprising any one of the following procedures:
a) dissolving Compound 1 in DCM, adding n-heptane in batches and stirring, to obtain Form U;
b) dissolving Compound 1 in the mixture of DCM/n-heptane (1:1, v/v) and stirring, to obtain Form U.

In some embodiments, Form A and/or Form B are obtained by the process of comprising adding a crystal seed in the solution system.

In some embodiments, the amorphous form is obtained by the process comprising any one of the following procedures:
a) dissolving Compound 1 in DCM, drying, to obtain the amorphous form, to obtain the amorphous form; or,
b) dissolving Compound 1 in a mixture of solvent containing DCM, drying, to obtain the amorphous form.

In some embodiments, the amorphous form is obtained by the process of comprising dissolving Compound 1 in a solid form, preferably a crystalline form of Compound 1.

### BRIEF DESCRIPTION OF THE FIGURES

FIG. 1A illustrates an X-ray powder diffraction (XRPD) pattern of Compound 1 Form A (EtOAc solvate 1:1) prepared according to Example 1A.

FIG. 1B illustrates a differential scanning calorimetry (DSC) profile of Compound 1 Form A prepared according to Example 1A.

FIG. 1C illustrates a thermogravimetric analysis (TGA) profile of Compound 1 Form A prepared according to Example 1A.

FIG. 1D illustrates a ¹H-nuclear magnetic resonance (¹H-NMR) spectrum of Compound 1 Form A (EtOAc solvate 1:1) prepared according to Example 1A.

FIG. 1E illustrates the calculated XRPD of the single crystal structure and the experimental XRPD of the single crystal of Compound 1 Form A.

FIG. 2A illustrates an X-ray powder diffraction (XRPD) pattern of Compound 1 Form B (anhydrate) prepared according to Example 2A.

FIG. 2B illustrates a differential scanning calorimetry (DSC)/thermogravimetric analysis (TGA) profile of Compound 1 Form B prepared according to Example 2A.

FIG. 2C illustrates a ¹H-nuclear magnetic resonance (¹H-NMR) spectrum of Compound 1 Form B prepared according to Example 2A.

FIG. 2D illustrates an XRPD overlay pattern of Compound 1 Form B prepared according to Example 2A before heating, heating to 120° C. and heating to 160° C.

FIG. 3A illustrates an X-ray powder diffraction (XRPD) pattern of Compound 1 Form C (MEK solvate) prepared according to Example 3A.

FIG. 3B illustrates a differential scanning calorimetry (DSC)/thermogravimetric analysis (TGA) profile of Compound 1 Form C prepared according to Example 3A.

FIG. 3C illustrates a ¹H-nuclear magnetic resonance (¹H-NMR) spectrum of Compound 1 Form C prepared according to Example 3A.

FIG. 4A illustrates an X-ray powder diffraction (XRPD) pattern of Compound 1 Form D (IPAc solvate) prepared according to Example 4A.

FIG. 4B illustrates a differential scanning calorimetry (DSC)/thermogravimetric analysis (TGA) profile of Compound 1 Form D prepared according to Example 4A.

FIG. 4C illustrates a ¹H-nuclear magnetic resonance (¹H-NMR) spectrum of Compound 1 Form D prepared according to Example 4A.

FIG. 5A illustrates an X-ray powder diffraction (XRPD) pattern of Compound 1 Form E (anisole solvate) prepared according to Example 5A.

FIG. 5B illustrates a differential scanning calorimetry (DSC)/thermogravimetric analysis (TGA) profile of Compound 1 Form E prepared according to Example 5A.

FIG. 5C illustrates a ¹H-nuclear magnetic resonance (¹H-NMR) spectrum of Compound 1 Form E prepared according to Example 5A.

FIG. 6A illustrates an X-ray powder diffraction (XRPD) pattern of Compound 1 Form F prepared according to Example 6A.

FIG. 6B illustrates a differential scanning calorimetry (DSC)/thermogravimetric analysis (TGA) profile of Compound 1 Form F prepared according to Example 6A.

FIG. 6C illustrates a ¹H-nuclear magnetic resonance (¹H-NMR) spectrum of Compound 1 Form F prepared according to Example 6A.

FIG. 7A illustrates an X-ray powder diffraction (XRPD) pattern of Compound 1 Form G prepared according to Example 7A.

FIG. 7B illustrates a differential scanning calorimetry (DSC)/thermogravimetric analysis (TGA) profile of Compound 1 Form G prepared according to Example 7A.

FIG. 7C illustrates a ¹H-nuclear magnetic resonance (¹H-NMR) spectrum of Compound 1 Form G prepared according to Example 7A.

FIG. 8A illustrates an X-ray powder diffraction (XRPD) pattern of Compound 1 Form H (anhydrate/hydrate) prepared according to Example 8A.

FIG. 8B illustrates a differential scanning calorimetry (DSC)/thermogravimetric analysis (TGA) profile of Compound 1 Form H prepared according to Example 8A.

FIG. 8C illustrates a ¹H-nuclear magnetic resonance (¹H-NMR) spectrum of Compound 1 Form H prepared according to Example 8A.

FIG. 9A illustrates an X-ray powder diffraction (XRPD) pattern of Compound 1 Form I (IPA solvate) prepared according to Example 9A.

FIG. 9B illustrates a differential scanning calorimetry (DSC)/thermogravimetric analysis (TGA) profile of Compound 1 Form I prepared according to Example 9A.

FIG. 9C illustrates a ¹H-nuclear magnetic resonance (¹H-NMR) spectrum of Compound 1 Form I prepared according to Example 9A.

FIG. 9D illustrates a differential scanning calorimetry (DSC)/thermogravimetric analysis (TGA) profile of Compound 1 Form I prepared according to Example 9B.

FIG. 9E illustrates a ¹H-nuclear magnetic resonance (¹H-NMR) spectrum of Compound 1 Form I prepared according to Example 9B.

FIG. 10A illustrates an X-ray powder diffraction (XRPD) pattern of Compound 1 Form J (2-MeTHF solvate) prepared according to Example 10A.

FIG. 10B illustrates a differential scanning calorimetry (DSC)/thermogravimetric analysis (TGA) profile of Compound 1 Form J prepared according to Example 10A.

FIG. 10C illustrates a ¹H-nuclear magnetic resonance (¹H-NMR) spectrum of Compound 1 Form J prepared according to Example 10A.

FIG. 11A illustrates an X-ray powder diffraction (XRPD) pattern of Compound 1 Form K (methyl acetate solvate) prepared according to Example 11 A.

FIG. 11B illustrates a differential scanning calorimetry (DSC)/thermogravimetric analysis (TGA) profile of Compound 1 Form K prepared according to Example 11A.

FIG. 11C illustrates a ¹H-nuclear magnetic resonance (¹H-NMR) spectrum of Compound 1 Form K prepared according to Example 11A.

FIG. 12A illustrates an X-ray powder diffraction (XRPD) pattern of Compound 1 Form L (anhydrate/hydrate) prepared according to Example 12A.

FIG. 12B illustrates a differential scanning calorimetry (DSC)/thermogravimetric analysis (TGA) profile of Compound 1 Form L prepared according to Example 12A.

FIG. 12C illustrates a ¹H-nuclear magnetic resonance (¹H-NMR) spectrum of Compound 1 Form L prepared according to Example 12A.

FIG. 13A illustrates an X-ray powder diffraction (XRPD) pattern of Compound 1 Form M (anhydrate) prepared according to Example 13A.

FIG. 13B illustrates a differential scanning calorimetry (DSC)/thermogravimetric analysis (TGA) profile of Compound 1 Form M prepared according to Example 13A.

FIG. 13C illustrates a ¹H-nuclear magnetic resonance (¹H-NMR) spectrum of Compound 1 Form M prepared according to Example 13A.

FIG. 14A illustrates an X-ray powder diffraction (XRPD) pattern of Compound 1 Form N (anhydrate) prepared according to Example 14A.

FIG. 14B illustrates a differential scanning calorimetry (DSC)/thermogravimetric analysis (TGA) profile of Compound 1 Form N prepared according to Example 14A.

FIG. 14C illustrates a ¹H-nuclear magnetic resonance (¹H-NMR) spectrum of Compound 1 Form N prepared according to Example 14A.

FIG. 15A illustrates an X-ray powder diffraction (XRPD) pattern of Compound 1 Form O (toluene solvate) prepared according to Example 15A.

FIG. 15B illustrates a differential scanning calorimetry (DSC)/thermogravimetric analysis (TGA) profile of Compound 1 Form O prepared according to Example 15A.

FIG. 15C illustrates a ¹H-nuclear magnetic resonance (¹H-NMR) spectrum of Compound 1 Form O prepared according to Example 15A.

FIG. 16A illustrates an X-ray powder diffraction (XRPD) pattern of Compound 1 Form P (chlorobenzene solvate) prepared according to Example 16A.

FIG. 16B illustrates a differential scanning calorimetry (DSC)/thermogravimetric analysis (TGA) profile of Compound 1 Form P prepared according to Example 16A.

FIG. 16C illustrates a ¹H-nuclear magnetic resonance (¹H-NMR) spectrum of Compound 1 Form Q prepared according to Example 16A.

FIG. 17A illustrates an X-ray powder diffraction (XRPD) pattern of Compound 1 Form Q (1,4-dioxane solvate) prepared according to Example 17A.

FIG. 17B illustrates a differential scanning calorimetry (DSC)/thermogravimetric analysis (TGA) profile of Compound 1 Form Q prepared according to Example 17A.

FIG. 17C illustrates a ¹H-nuclear magnetic resonance (¹H-NMR) spectrum of Compound 1 Form Q prepared according to Example 17A.

FIG. 18A illustrates an X-ray powder diffraction (XRPD) pattern of Compound 1 Form R (anhydrate/hydrate) prepared according to Example 18A.

FIG. 18B illustrates a differential scanning calorimetry (DSC)/thermogravimetric analysis (TGA) profile of Compound 1 Form R prepared according to Example 18A.

FIG. 18C illustrates a ¹H-nuclear magnetic resonance (¹H-NMR) spectrum of Compound 1 Form R prepared according to Example 18A.

FIG. 19A illustrates an X-ray powder diffraction (XRPD) pattern of Compound 1 Form S prepared according to Example 19A.

FIG. 19B illustrates a differential scanning calorimetry (DSC)/thermogravimetric analysis (TGA) profile of Compound 1 Form S prepared according to Example 19A.

FIG. 20 illustrates an X-ray powder diffraction (XRPD) pattern of Compound 1 Form T prepared according to Example 19A.

FIG. 21A illustrates an X-ray powder diffraction (XRPD) pattern of Compound 1 Form U (anhydrate) prepared according to Example 21A.

FIG. 21B illustrates a differential scanning calorimetry (DSC) profile of Compound 1 Form U prepared according to Example 21A.

FIG. 21C illustrates a thermogravimetric analysis (TGA) profile of Compound 1 Form U prepared according to Example 21A.

FIG. 21D illustrates a ¹H-nuclear magnetic resonance (¹H-NMR) spectrum of Compound 1 Form U prepared according to Example 21.

FIG. 22A illustrates an X-ray powder diffraction (XRPD) pattern of Compound 1 amorphous Form.

FIG. 22B illustrates a differential scanning calorimetry (DSC)/thermogravimetric analysis (TGA) profile of Compound 1 in the amorphous form.

FIG. 23 illustrates the interconversions of the crystalline forms of Compound 1.

### DETAILED DESCRIPTION OF THE DISCLOSURE

Unless defined otherwise, all technical and scientific terms used herein have the same meaning as is commonly understood by one of skill in the art to which this invention belongs. All patents, patent applications, and publications referred to herein are incorporated by reference.

As used herein, the term “solvate” refers to a crystalline form of Compound 1 which contains solvent.

As used herein, the term “subject,” “individual,” or “patient,” used interchangeably, refers to any animal, including mammals such as mice, rats, other rodents, rabbits, dogs, cats, swine, cattle, sheep, horses, primates, and humans. In some embodiments, the patient is a human. In some embodiments, the subject has experienced and/or exhibited at least one symptom of the disease or disorder to be treated and/or prevented. In some embodiments, the subject is suspected of having a multi-tyrosine kinase-associated cancer.

As used herein, a “therapeutically effective amount” of a crystalline form of a salt of Compound 1 is an amount that is sufficient to ameliorate, or in some manner reduce a symptom or stop or reverse the progression of a condition, or negatively modulate or inhibit the activity of a multi-tyrosine kinase. Such amount may be administered as a single dosage or may be administered according to a regimen, whereby it is effective.

As used herein, term “form” is used to described the a crystalline form, which is interchangeable with term “type”. The term “crystal form” or “crystalline form” refers to a solid form that is crystalline. In certain embodiments, a crystal form of a substance may be substantially free of amorphous forms and/or other crystal forms. In certain embodiments, a crystal form of a substance may contain less than about 1%, less than about 2%, less than about 3%, less than about 4%, less than about 5%, less than about 6%, less than about 7%, less than about 8%, less than about 9%, less than about 10%, less than about 15%, less than about 20%, less than about 25%, less than about 30%, less than about 35%, less than about 40%, less than about 45%, or less than about 50% by weight of one or more amorphous forms and/or other crystal forms. In certain embodiments, a crystal form of a substance may be physically and/or chemically pure. In certain embodiments, a crystal form of a substance may be about 99%, about 98%, about 97%, about 96%, about 95%, about 94%, about 93%, about 92%, about 91%, or about 90% physically and/or chemically pure.

As used herein, an “amorphous form” refers to a particle without definite structure, such as lacking crystalline structure. Unless otherwise specified, the term “amorphous” or “amorphous form” means that the substance, component, or product in question is not substantially crystalline as determined by X-ray diffraction. In particular, the term “amorphous form” describes a disordered solid form, i.e., a solid form lacking long range crystalline order. In certain embodiments, an amorphous form of a substance may be substantially free of other amorphous forms and/or crystal forms. In certain embodiments, an amorphous form of a substance may contain less than about 1%, less than about 2%, less than about 3%, less than about 4%, less than about 5%, less than about 10%, less than about 15%, less than about 20%, less than about 25%, less than about 30%, less than about 35%, less than about 40%, less than about 45%, or less than about 50% by weight of one or more other amorphous forms and/or crystal forms on a weight basis. In certain embodiments, an amorphous form of a substance may be physically and/or chemically pure. In certain embodiments, an amorphous form of a substance be about 99%, about 98%, about 97%, about 96%, about 95%, about 94%, about 93%, about 92%, about 91%, or about 90% physically and/or chemically pure.

As used herein, “treatment” means any manner in which the symptoms or pathology of a condition, disorder or disease are ameliorated or otherwise beneficially altered. Treatment also encompasses any pharmaceutical use of the compositions herein.

As used herein, amelioration of the symptoms of a particular disorder by administration of a particular pharmaceutical composition refers to any lessening, whether permanent or temporary, lasting or transient that can be attributed to or associated with administration of the composition.

As used herein, the term “about” when used in reference to XRPD peak positions refers to the inherent variability of peaks depending on the calibration of the instrument, processes used to prepare the crystalline forms of the present invention, age of the crystalline forms and the type of instrument used in the analysis. The variability of the instrumentation used for XRPD analysis was about ±0.1° 2θ.

As used herein, the term “about” when used in reference to DSC endothermic peak onset refers to the inherent variability of peaks depending on the calibration of the instrument, method used to prepare the samples of the present invention, and the type of instrument used in the analysis. The variability of the instrumentation used for DSC analysis was about ±1° C.

### General Methods

The general methods outlined below were used in the exemplified Examples unless otherwise noted.

### I. Crystallization Techniques

Crystalline forms disclosed herein may be prepared using a variety of methods well known to those skilled in the art including crystallization or recrystallization from a suitable solvent or by sublimation. A wide variety of techniques may be employed, including those in the exemplified Examples, for crystallization or recrystallization including evaporation of a water-miscible or a water-immiscible solvent or solvent mixture, crystal seeding in a supersaturated solution, decreasing the temperature of the solvent mixture, or freeze drying the solvent mixture.

Crystallization disclosed herein may be done with or without crystal seed. The crystal seed may come from any previous batch of the desired crystalline form such as Form C, Form D, Form E, Form F, Form G, Form H, Form I, Form J, Form K, Form L, Form M, Form N, Form O, Form P, Form Q, Form R, Form S or Form T.

ABBREVIATIONS and ACRONYMS

| | Abbreviations/ | |
|---|---|---|
| Category | Acronyms | Full Name/Description |
| Analytical | DSC | Differential scanning calorimetry |
| Techniques | DVS | Dynamic vapor sorption |
| | HPLC | High Performance Liquid |
| | | Chromatography |
| | NMR | Nuclear magnetic resonance |
| | TGA | Thermogravimetric analysis |
| | XRPD | X-ray powder diffraction |
| Solvent | MeOH | Methanol |
| | 2-MeTHF | 2-Methyltetrahydrofuran |
| | ACN | Acetonitrile |
| | CHCl3 | Trichloromethane |
| | CPME | Cyclopentyl Methyl Ether |
| | DCM | Dichloromethane |
| | DMF | N,N-Dimethylformamide |
| | DMSO | Dimethylsulfoxide |
| | EtOAc, EA | Ethyl acetate |
| | EtOH | Ethanol |
| | IPA | Isopropyl alcohol |
| | IPAc | Isopropyl acetate |
| | MEK | Methyl ethyl ketone |
| | MIBK | Methyl isobutyl ketone |
| | MTBE | Methyl tert-butyl ether |
| | NMP | N-Methyl pyrrolido |
| | THF | Tetrahydrofuran |
| Agent | t-BuOK | Potassium tert-butoxide |
| | EDSA | 1,2-ethanedisulfonic acid |
| | NaOH | Sodium hydroxide |
| Others | aq. | Aqueous |
| | BE | Birefringence with Extinction |
| | Eq. | Equivalent |
| | IPC | In process control |
| | Vol or vol. | Volume |
| | w/w | Weight / weight |

### Instruments and Parameters

For XRPD analysis, a PANalytical Empyrean and X′ Pert3 X-ray powder diffractometer were used to characterize the physical forms obtained in the present disclosure, without special instructions. The XRPD parameters used are listed as follows.

| Parameters | XRPD | |
|---|---|---|
| Model | Empyrean | X′ Pert³ |
| X-Ray wavelength | Cu, kα, | Cu, kα, |
| | Kα1 (Å): 1.540598, | Kα1 (Å): 1.540598, |
| | Kα2 (Å): 1.544426 | Kα2 (Å): 1.544426 |
| | Kα2/Kα1 intensity | Kα2/Kα1 intensity |
| | ratio: 0.50 | ratio: 0.50 |
| X-Ray tube setting | 45 kV, 40 mA | 45 kV, 40 mA |
| Divergence slit | Automatic | 1/8° |
| Scan mode | Continuous | Continuous |
| Scan range (°2Theta) | 3-40 | 3-40 |
| Scan step time (s) | 17.8 | 46.7 |
| Step size (°2TH) | 0.0167 | 0.0263 |
| Test Time (s) | 5 min 30 s | About 5 mins (5 min 04 s) |

For XRPD analysis, a Bruker D8 advanced X-Ray Powder diffractometer or equivalent was also used to characterize Form A and Form U. The XRPD parameters used are listed as follows.

| | |
|---|---|
| Tube | Cu, kα, Kα (Å): 1.540598, |
| Generator | Voltage: 40 kV; Current: 40 mA |
| Fixed incident beam optics | Primary Soller slit: 2.5 deg |
| | Secondary Soller slit: 2.5 deg |
| | Divergence Slit: 0.60 mm |
| | Slit: fixed |
| Detector | Detector Name: Lynxeye (1 D mode) |
| | PSD OPENING 2.1o |
| Scan mode | Continuous PSD fast |
| Scan range (°2Theta) | 4-40 deg |
| Scan type | Coupled Two Theta/Theta |
| Increment | 0.02 deg |
| Time/step | 0.12 s/step |

TGA and DSC were used to characterize the physical forms obtained in the present disclosure, without special instructions, wherein TGA data were collected using a TA Q500/Q5000 TGA from TA Instruments; and, DSC was performed using a TA Q200/Q2000 DSC from TA Instruments. Detailed parameters used are listed as follows.

| Parameters | TGA | DSC | mDSC |
|---|---|---|---|
| Method | Ramp | Ramp | Modulated |
| Sample pan | Aluminum, | Aluminum, | Aluminum, |
| | open | crimped | crimped |
| Temperature | RT - | 25° C. - | 16° C. - |
| | desired | desired | desired |
| | temperature | temperature | temperature |
| Heating rate | 10° C./min | 10° C./min | 3° C./min |
| Purge gas | N2 | N2 | N2 |

For TGA and DGA analysis of Form A or U, some instruments were also used to conduct the testing, wherein TGA data were collected using a NETZSCH TG 209 F1 Instruments; and, DSC was performed using a TA Q 20 or TA DSC 250 Instruments. Detailed parameters used are listed as follows.

| Parameters | TGA | DSC |
|---|---|---|
| Method | Ramp | Ramp |
| Sample pan | Aluminum, open | Aluminum, Sealed |
| Temperature | RT - desired | RT - desired |
| | temperature | temperature |
| Heating rate | 10° C./min | 10° C./min |
| Purge gas | N2 | N2 |

DVS of the obtained forms in the present disclosure was measured via an SMS (Surface Measurement Systems) DVS Intrinsic, without special instructions (Method A). The relative humidity at 25° C. was calibrated against deliquescence point of LiCl, Mg(NO3)2 and KCl. Parameters for the DVS test are listed as follows.

| Parameters | DVS |
|---|---|
| Temperature | 25° C. |
| Sample size | 10~20 mg |
| Gas and flow rate | N2, 200 mL/min |
| dm/dt | 0.002%/min |
| Min. dm/dt stability | 10 min |
| duration | |
| Max. equilibrium time | 180 min |
| RH range | 70% RH-95% RH-0% RH-95% RH |
| RH step size | 10% (0% RH-90% RH, 90% RH-0% RH) |
| | 5% (90% RH-95% RH, 95% RH-90% RH) |

DVS of Form A and U was also measured via an SMS (Surface Measurement Systems) DVS Intrinsic (Method B). The relative humidity at 25° C. was calibrated against deliquescence point of LiCl, Mg(NO3)2, and KCL. Parameters for DVS test are listed as follows.

| Parameters | DVS |
|---|---|
| Temperature | 25° C. |
| Gas and flow rate | N2, 200 mL/min |
| dm/dt | <0.01 min |
| Min. dm/dt stability duration | 60 min |
| Max. equilibrium time | 180 min |
| RH range | Cycle: 40%-0%-95%-0%-40% RH |

The single crystal X-ray diffraction data were collected at 120 K using Rigaku XtaLAB Synergy R (CuK radiation, 1.54184 Å) diffractometer. The instrument parameters are listed as follows.

| | |
|---|---|
| Instrument | Rigaku XtaLAB Synergy R |
| X-Ray sources generator | MicroMax-007HF X-ray source |
| | (Cu/kα: 1.54184 Å) |
| Detector | HyPix 6000HE detector |
| Goniometer | Four-circle Kappa Goniometer |
| Low Temperature Devices | Cryostream-700 |
| | (Oxford Cryosystems) |
| Software package | CrysAlisPro (V1.171.40.14e) |

The following Examples are intended to illustrate further certain embodiments of the invention and are not intended to limit the scope of the invention.

### EXAMPLE

Methods for manufacturing the Bcl-2 inhibitor 2-((1H-pyrrolo[2,3-b]pyridin-5-yl)oxy)-N-((4-((((1r,4r)-4-hydroxy-4-methylcyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)benzamide (Compound 1), are known. For example, international Publication No. WO2019/210828 provides a detailed synthetic route for the preparation of Compound 1.

### EXAMPLE 1A: Preparation of Compound 1 Form A (Form A)

Compound 1 (40 g) was dissolved in DCM (120 mL). After concentrating the solution to dry, EA (250 mL) was added. The resulting mixture was warmed to 60-70° C., slowly cooled to 15-25° C., and then filtered. The resulting cake was dried for 16 hours at 40-50° C. to give compound 1 Form A (about 40 g), which could be used as a crystal seed.

Compound 1 (8.1 kg) was dissolved in DCM (58 kg) at 20-30° C. After concentrating the solution to about half the volume of the mixture, EA (45 kg) was charged to the solution, and a crystal seed (0.035 kg) was added. After stirring for 1 hour at 20-30° C., the solution was concentrated to exchange EA solvent mixture three times with EA (43 kg+43 kg+24 kg). The mixture was heated to 60-70° C. and stirred for 2 hours, and then slowly cooled to 15-25° C.

MeOH (32 kg) was introduced to the resulting mixture at 45-55° C. and stirred for 16 hours. After solvent exchanging with MeOH (20 kg+21 kg+20 kg) three times, the mixture was returned to EA solution by exchanging with EA (23 kg+47 kg+40 kg) three times. The mixture was warmed to 60-70° C. and stirred for 2.5 hours and then slowly cooled to 15-25° C. The resulting mixture was slowly cooled to 15-25° C. and filtered. The resulting cake was washed with EA (9 kg) and dried at 45-55° C. for 18.5 hours, to give a product as yellow solid. After sieving the solid, a total of 7.36 kg of Compound Form A was obtained.

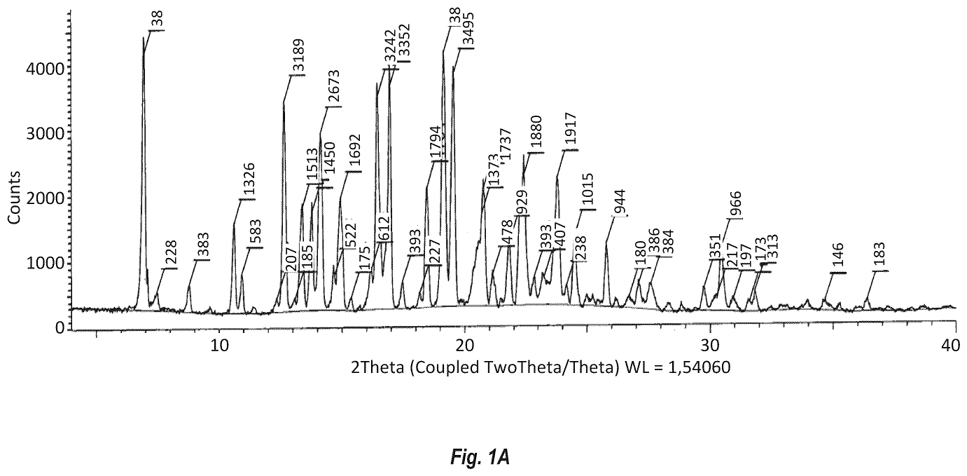

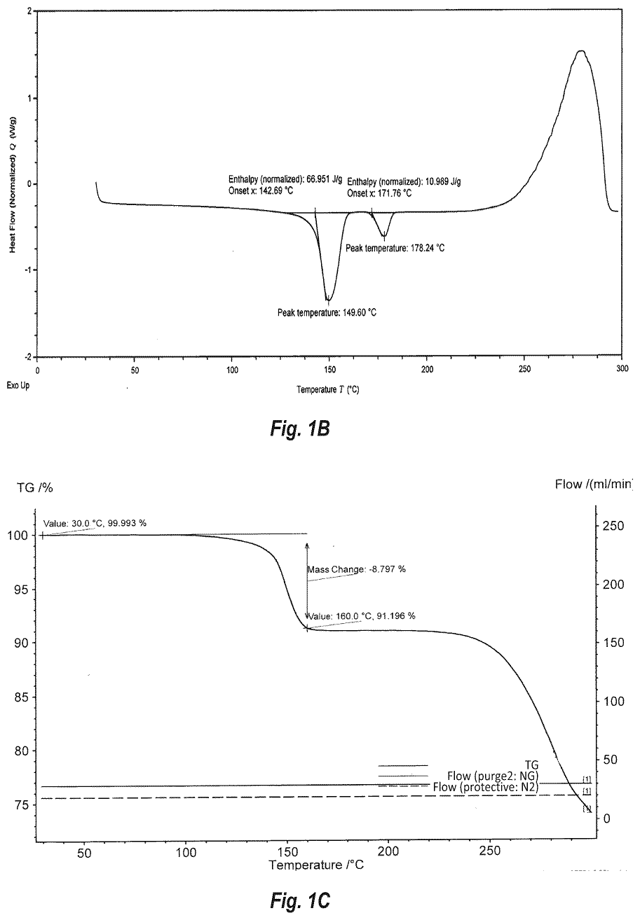

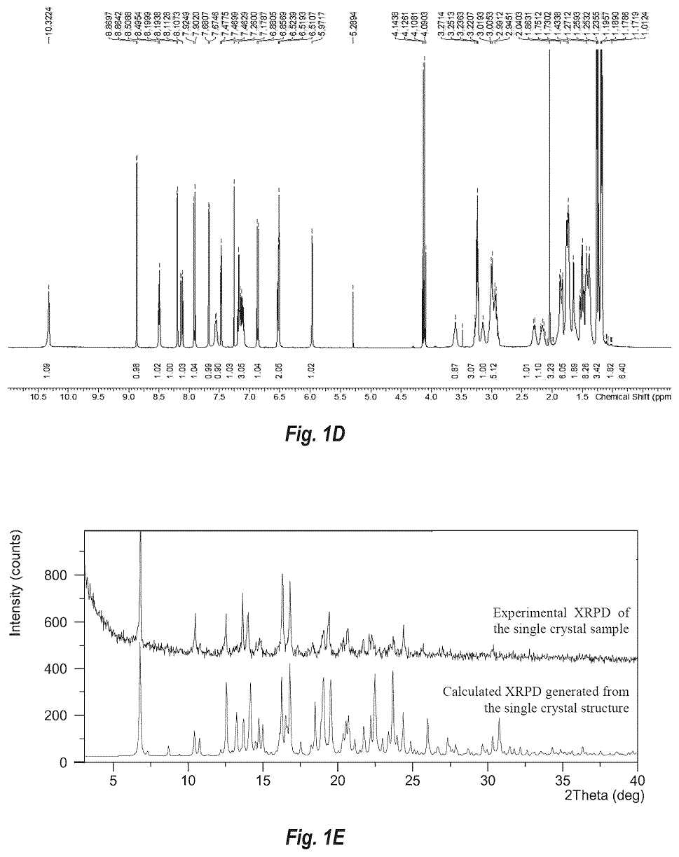

The X-ray powder diffraction (XRPD) pattern (conducted on Bruker D8 advanced X-Ray Powder diffractometer) was used to characterize the obtained Form A, which showed that Form A was in a crystalline form, see FIG. 1A. The characteristic peaks and percent peak intensities obtained from the XRPD analysis are listed in Table 1A.

TABLE 1A
XRPD pattern of Compound 1 Form A

| Pos. [°2θ] | d-spacing [Å] | Rel. Int. [%] |
|---|---|---|
| 6.928 | 12.74962 | 199.9 |
| 7.431 | 11.88697 | 5.9 |
| 8.762 | 10.08430 | 9.9 |
| 10.603 | 8.33666 | 34.4 |
| 10.919 | 8.09612 | 15.1 |
| 12.359 | 7.15614 | 5.4 |
| 12.654 | 6.98993 | 182.8 |
| 13.090 | 6.75786 | 4.8 |
| 13.363 | 6.62063 | 39.3 |
| 13.760 | 6.43061 | 37.7 |
| 14.126 | 6.26474 | 69.4 |
| 14.701 | 6.02105 | 13.5 |
| 14.936 | 5.92683 | 43.9 |
| 15.350 | 5.76762 | 4.6 |
| 16.197 | 5.46788 | 15.9 |
| 16.456 | 5.38232 | 84.2 |
| 16.928 | 5.23335 | 87.0 |
| 17.455 | 5.07668 | 10.2 |
| 18.179 | 4.87613 | 5.9 |
| 18.456 | 4.80356 | 46.6 |
| 19.142 | 4.63277 | 100.0 |
| 19.524 | 4.54294 | 90.8 |
| 20.693 | 4.28895 | 35.7 |
| 20.737 | 4.27996 | 45.1 |
| 21.144 | 4.19839 | 12.4 |
| 21.796 | 4.07435 | 24.1 |
| 22.380 | 3.96928 | 48.8 |
| 22.837 | 3.89098 | 10.2 |
| 23.251 | 3.82265 | 10.6 |
| 23.785 | 3.73795 | 49.8 |
| 24.123 | 3.68627 | 6.2 |
| 24.497 | 3.63087 | 26.4 |
| 25.791 | 3.45163 | 24.5 |
| 26.719 | 3.33379 | 4.7 |
| 27.108 | 3.28677 | 10.0 |
| 27.592 | 3.23023 | 10.0 |
| 29.751 | 3.00059 | 9.1 |

¹H NMR spectrum of Compound From A was shown in FIG. 1D. DSC/TGA curve (conducted on NETZSCH TG 209 F1 Instrument and TA Q 20) showed that a weight loss of 8.8% up to 160° C. and two endotherm peaks at 149.6° C. and 178.2° C. (peak) were observed (FIG. 1B and FIG. 1C). XRPD overlay showed that Form A converted to From B after heating to 160° C., cooling back to RT and re-exposed to air conditions. Combined with TGA data and ¹H NMR results, Form A was speculated as an EtOAc solvate.

Compound 1 Form A was stepwise isothermal by TGA in a nitrogen atmosphere. When the weight loss reaches 0.02%, the system equilibrated at a certain temperature till weight loss <0.002%. The results showed that after heating From A stepwise to 100° C., the TGA weight loss matched the weight loss detected by linear heating. After cooling back to RT, Form B of low crystallinity was obtained.

The DVS cycle was conducted at 25° C. (Method B), the sorption and desorption were revisable during the full DVS cycle, the water sorption is 0.4% at 95% RH humidity, the Compound 1 form A is slightly hygroscopic.

### EXAMPLE 1B: Preparation of Compound 1 Form A

Compound 1 (7.0 g) was added into EA (140 mL) then was heated to reflux for 2 hours. The mixture was slowly cooled down to room temperature (RT) and stirred for 0.5 hours, filtrated, washed with EA and dried over reduced pressure, to give the product (4.9 g).

### EXAMPLE 1C: Preparation of Compound 1 Form A in Single Crystal

Compound 1 (2.8 mg) was dissolved in 0.5 mL THF/EtOAc (1:2, v/v) solvent mixture. After slow evaporation, the single crystals of Compound 1 Form A were obtained.

The single crystal of Compound 1 Form A (EtOAc solvate) was characterized by SCXRD. The calculated XRPD of the single crystal structure is nearly consistent with the experimental XRPD of the single crystal of Form A (FIG. 1E).

The single crystal was analyzed by single-crystal X-ray diffractometer. The crystal system of the single crystal is triclinic and the space group is P1. The cell parameters are: {a=13.64421(4) Å, b=14.07005(4) Å, c=15.01208(4) Å, α=112.0202(3) °, β=104.6821(3) °, γ=93.6507(2) °, V=2543.673(14) Å3}.

The asymmetric unit of the single crystal structure is comprised of two Compound 1 molecules and two EtOAc molecules, which indicates that the crystal is an EtOAc solvate and the molar ratio of Compound 1 to EtOAc is 1:1. And, adjacent Compound 1 molecules connect with each other through intermolecular hydrogen bonds.

### EXAMPLE 2A: Preparation of Compound 1 Form B

Compound 1 (20 mg) was dissolved in acetone. The mixtures were filtered, and then the obtained clear solution was subjected to slow evaporation at RT, to obtain Form B.

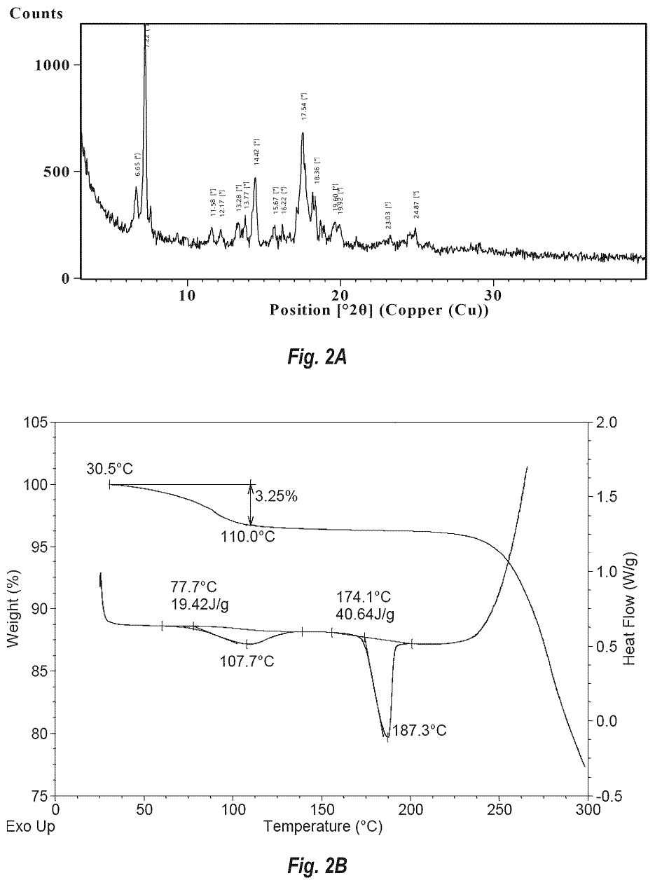

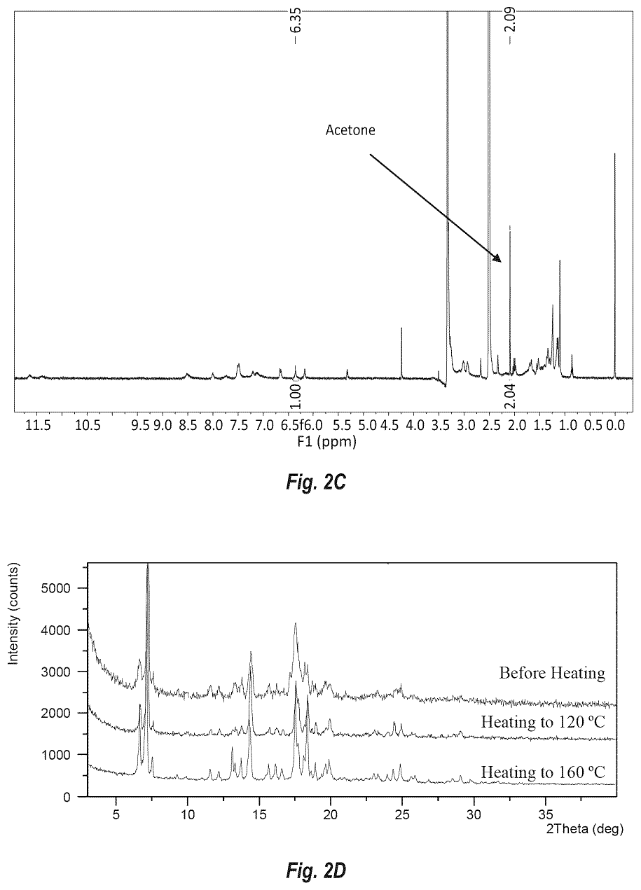

The XRPD pattern was used to characterize the obtained Form B which showed that Form B was in a crystalline, see FIG. 2A. The characteristic peaks and percent peak intensities obtained from the XRPD analysis are listed in Table 2A.

TABLE 2A
XRPD pattern of Compound 1 Form B

| Pos. [°2θ] | d-spacing [Å] | Rel. Int. [%] |
|---|---|---|
| 6.65 | 13.30 | 21.12 |
| 7.22 | 12.24 | 100.00 |
| 11.58 | 7.64 | 7.80 |
| 12.17 | 7.27 | 7.39 |
| 13.28 | 6.67 | 9.80 |
| 13.77 | 6.43 | 11.34 |
| 14.42 | 6.14 | 32.09 |
| 15.67 | 5.66 | 8.98 |
| 16.22 | 5.47 | 8.91 |
| 17.54 | 5.06 | 54.27 |
| 18.36 | 4.83 | 23.45 |
| 19.60 | 4.53 | 10.71 |
| 19.92 | 4.46 | 9.02 |
| 23.03 | 3.86 | 3.35 |
| 24.87 | 3.58 | 8.92 |

The TGA/DSC curve showed that a weight loss of 3.3% up to 110° C. and two endothermic peaks at 107.7° C. and 187.3° C. (peak) before decomposition were detected (FIG. 2B). In the ¹H NMR spectrum, about 2.2% of acetone was observed (FIG. 2C). After heating Form B to 160° C., no form change was observed.

Results of VT-XRPD displayed that after heating From B to 150° C. and cooling back to 30° C. in N2 atmosphere, no form change was observed, which indicated Form B was an anhydrate. The acetone detected in ¹H NMR was speculated to be caused by solvent residual.

In addition, Form B with high crystallinity could be obtained after heating Form B to 160° C., cooling back to RT and then heating to 160° C. again, see FIG. 2D. The TGA/DSC curve showed a weight loss of 2.8% up to 150° C. and one endothermic peak at 186.5° C. (peak) before decomposition was observed. And no signal of acetone was detected in ¹H NMR spectrum.

### EXAMPLE 2B: Preparation of Compound 1 Form B

Compound 1 Form B was obtained by any one of the following steps:
1) heating Form A to 160° C. and then naturally cool back to RT;
2) heating Form A stepwise isothermally to 100° C.;
3) heating Form D to 130° C. and being isothermal for 30 mins; or,
4) heating Form J to 130° C. and being isothermal for 30 mins.
5) heating Form B to 160° C. and cool back to RT, then heating to 160° C.

### EXAMPLE 2C: Preparation of Compound 1 Form B

Compound 1 Form K (6.0 g) was added into heptane (100 mL) then was refluxed at a temperature of about 100° C., and subjected to slurry for 24 h. The mixture was cooled down to RT and filtrated, washed with heptane and dried over reduced pressure to give the product (5.5 g).

### EXAMPLE 3A: Preparation of Compound 1 Form C (Form C)

Compound 1 (20 mg) was dissolved in MEK. The mixtures were filtered, and then the obtained clear solution was subjected to slow evaporation at RT, to obtain Form C.

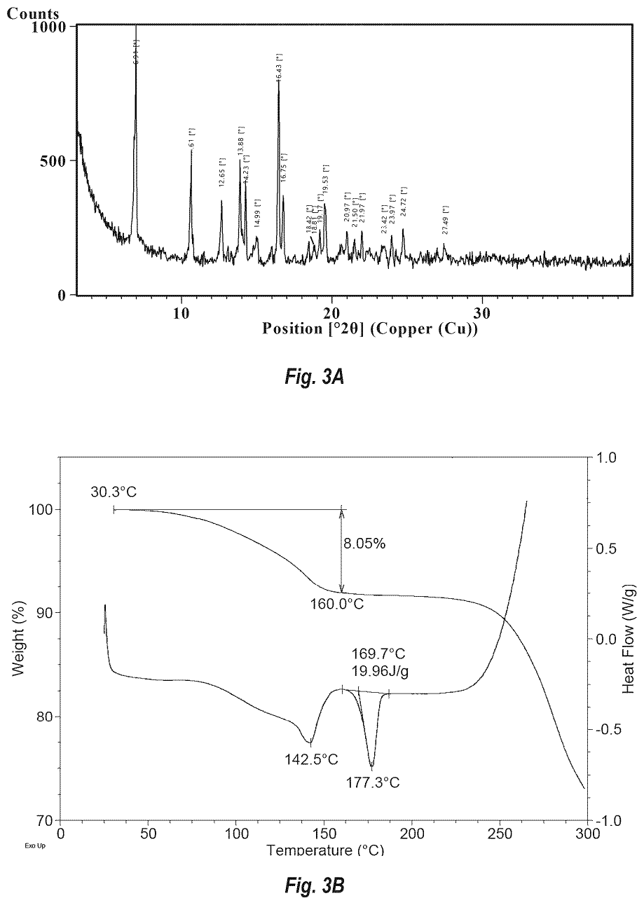

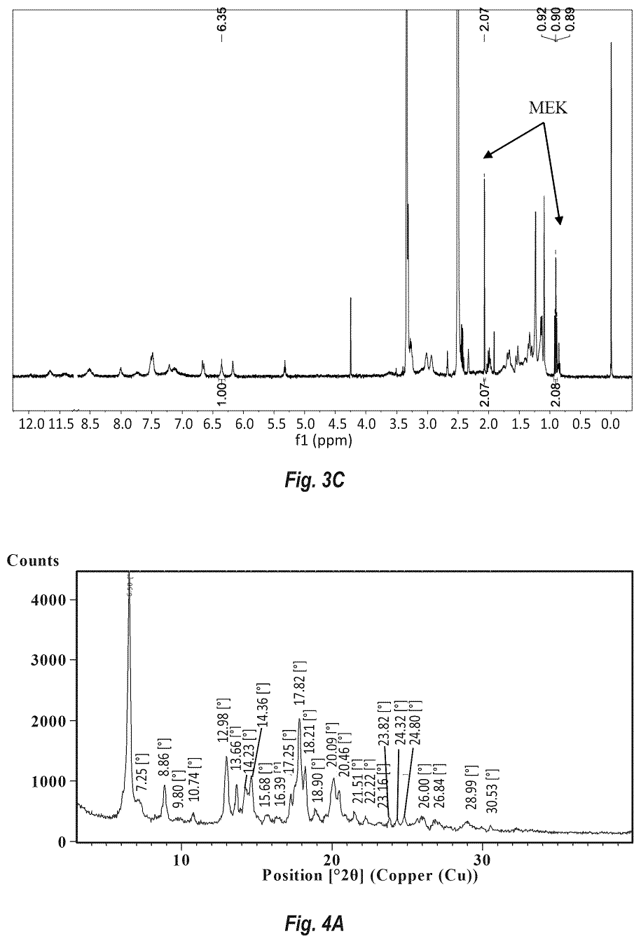

The X-ray powder diffraction (XRPD) pattern was used to characterize the obtained Form C, which showed that Form C was in a crystalline form, see FIG. 3A. The characteristic peaks and percent peak intensities obtained from the XRPD analysis are listed in Table 3A.

TABLE 3A
XRPD pattern of Compound 1 Form C

| Pos. [°2θ] | d-spacing [Å] | Rel. Int. [%] |
|---|---|---|
| 6.91 | 12.79 | 100.00 |
| 10.61 | 8.34 | 53.40 |
| 12.65 | 7.00 | 36.44 |
| 13.88 | 6.38 | 55.60 |
| 14.23 | 6.22 | 38.85 |
| 14.99 | 5.91 | 12.75 |
| 16.43 | 5.39 | 99.90 |
| 16.75 | 5.29 | 39.88 |
| 18.42 | 4.82 | 9.40 |
| 18.81 | 4.72 | 8.65 |
| 19.17 | 4.63 | 15.78 |
| 19.53 | 4.55 | 33.14 |
| 20.97 | 4.24 | 16.62 |
| 21.50 | 4.13 | 13.42 |
| 21.97 | 4.05 | 16.02 |
| 23.42 | 3.80 | 8.88 |
| 23.97 | 3.71 | 14.93 |
| 24.72 | 3.60 | 19.55 |
| 27.49 | 3.25 | 7.94 |

TGA/DSC curve showed a weight loss of 8.1% up to 160° C. and two endotherm peaks at 142.5° C. and 177.3° C. (peak) (FIG. 3B). ¹H NMR spectrum (FIG. 3C) showed that the theoretical weight of MEK was calculated as 5.4%, which was lower than TGA weight loss and was speculated to be caused by solvent loss during storage before ¹H NMR test. To figure out whether the weight loss was solvent absorption or not, heating experiments were performed on Form C.

XRPD comparison showed that after heating to 160° C., cooling back to RT and re-exposed at air conditions, Form C converted to Form B with weak crystallinity. Form C was speculated as a MEK solvate.

### EXAMPLE 4A: Preparation of Compound 1 Form D (Form D)

Amorphous Compound 1 (20 mg) was suspended in IPAc. The suspension was subjected to slurry at RT by stirring for 1˜7 days, to obtain Form D.

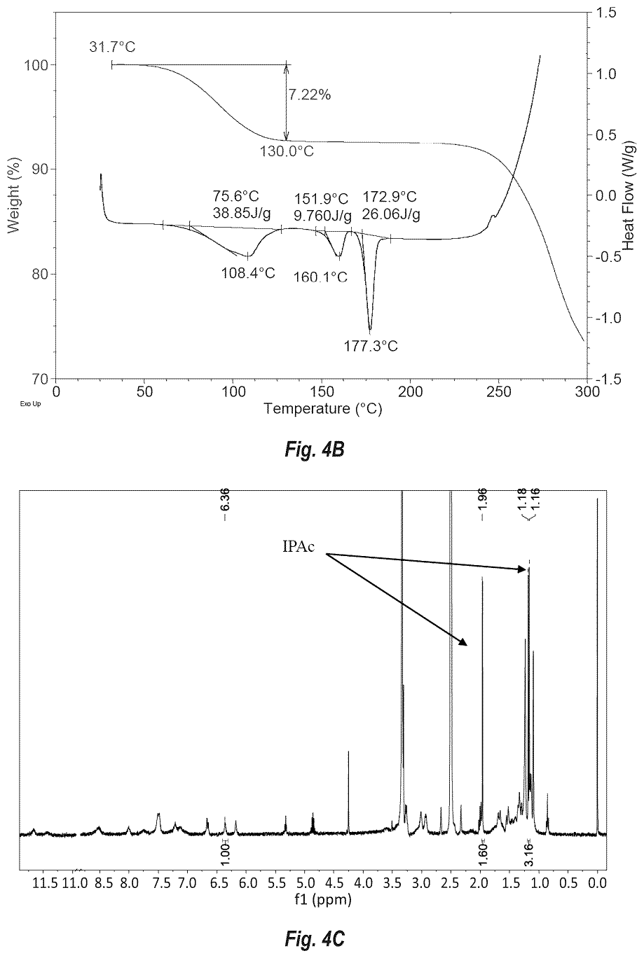

The X-ray powder diffraction (XRPD) pattern was used to characterize the obtained Form D, which showed that Form D was in a crystalline form, see FIG. 4A. The characteristic peaks and percent peak intensities obtained from the XRPD analysis are listed in Table 4A.

TABLE 4A
XRPD pattern of Compound 1 Form D

| Pos. [°2θ] | d-spacing [Å] | Rel. Int. [%] |
|---|---|---|
| 6.50 | 13.59 | 100.00 |
| 7.25 | 12.20 | 6.55 |
| 8.86 | 9.99 | 14.43 |
| 9.80 | 9.02 | 0.98 |
| 10.74 | 8.24 | 3.12 |
| 12.98 | 6.82 | 27.15 |
| 13.66 | 6.48 | 15.70 |
| 14.23 | 6.23 | 14.18 |
| 14.63 | 6.06 | 19.43 |
| 15.68 | 5.65 | 3.95 |
| 16.39 | 5.41 | 2.92 |
| 17.25 | 5.14 | 12.56 |
| 17.82 | 4.98 | 43.14 |
| 18.21 | 4.87 | 23.91 |
| 18.90 | 4.70 | 6.63 |
| 20.09 | 4.42 | 19.12 |
| 20.46 | 4.34 | 14.52 |
| 21.51 | 4.13 | 5.51 |
| 22.22 | 4.00 | 4.07 |
| 23.16 | 3.84 | 2.14 |
| 23.83 | 3.73 | 4.03 |
| 24.32 | 3.66 | 3.56 |
| 24.80 | 3.59 | 4.84 |
| 26.00 | 3.43 | 4.53 |
| 26.84 | 3.32 | 3.09 |
| 28.99 | 3.08 | 3.23 |

The TGA/DSC data displayed a weight loss of 7.2% up to 130° C., three endothermic peaks at 108.4° C., 160.1° C., and 177.3° C. (FIG. 4B). ¹H NMR spectrum showed that the theoretical content of IPAc was determined as 5.4%, indicating that there might be some solvent loss during the storage (FIG. 4C).

In the XRPD overlay of the heating experiment, form change was observed for Form D after heating to 165° C. and cooling back to RT. Hence, Form D was speculated as an IPAc solvate.

In addition, the results of the heating experiment showed that after heating Form D to 130° C. and being isothermal for 30 min, Form B in low crystallinity with an extra peak was obtained.

### EXAMPLE 5A: Preparation of Compound 1 Form E (Form E)

Compound 1 (20 mg) was dissolved in anisole. The mixtures were filtered, and then the obtained clear solution was subjected to slow evaporation at RT, to obtain Form E.

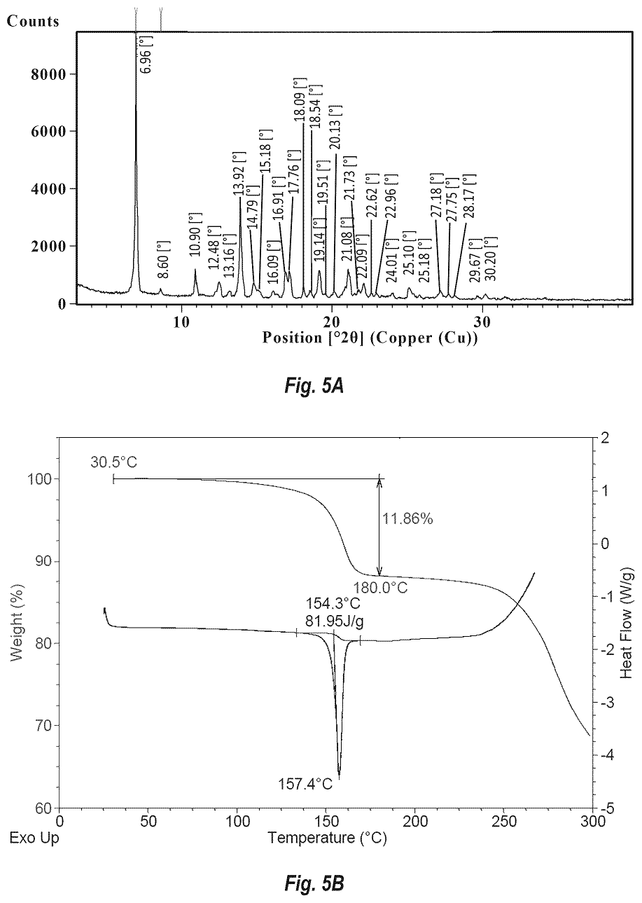

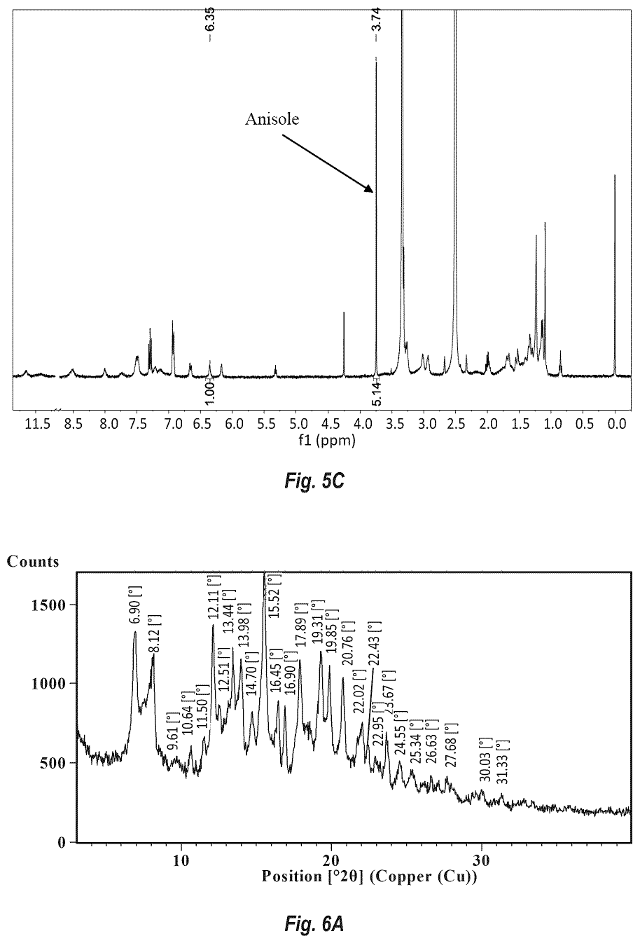

The X-ray powder diffraction (XRPD) pattern was used to characterize the obtained Form E, which showed that Form E was in a crystalline form, see FIG. 5A. The characteristic peaks and percent peak intensities obtained from the XRPD analysis are listed in Table 5A.

TABLE 5A
XRPD pattern of Compound 1 Form E

| Pos. [°2θ] | d-spacing [Å] | Rel. Int. [%] |
|---|---|---|
| 6.96 | 12.70 | 100.00 |
| 8.60 | 10.29 | 1.95 |
| 10.90 | 8.12 | 11.86 |
| 12.48 | 7.09 | 6.29 |
| 13.16 | 6.73 | 2.24 |
| 13.91 | 6.37 | 38.41 |
| 14.79 | 5.99 | 6.37 |
| 15.18 | 5.84 | 2.37 |
| 16.09 | 5.51 | 2.66 |
| 16.91 | 5.24 | 10.97 |
| 17.16 | 5.17 | 11.50 |
| 18.09 | 4.90 | 4.50 |
| 18.54 | 4.78 | 2.90 |
| 19.14 | 4.64 | 11.69 |
| 19.51 | 4.55 | 1.34 |
| 20.13 | 4.41 | 2.79 |
| 21.08 | 4.21 | 10.74 |
| 21.73 | 4.09 | 3.29 |
| 22.09 | 4.02 | 6.11 |
| 22.62 | 3.93 | 2.01 |
| 22.96 | 3.87 | 2.07 |
| 24.01 | 3.71 | 2.14 |
| 25.10 | 3.55 | 4.72 |
| 25.81 | 3.45 | 1.59 |
| 27.18 | 3.28 | 3.39 |
| 27.75 | 3.21 | 2.10 |
| 28.17 | 3.17 | 1.39 |
| 29.67 | 3.01 | 1.15 |

TGA curve showed a weight loss of 11.9% up to 180° C. and DSC curve showed one endothermic peak at 157.4° C. (peak) before decomposition (FIG. 5B). Based on the ¹H NMR spectrum (FIG. 5C), about 17.1% anisole was determined, which was higher than the TGA weight loss and was speculated to be caused by the inhomogeneous solvent residual. Results of the heating experiment showed that a decrease of crystallinity was observed after heating Form E to 170° C. and then cooling back, indicating the endothermic peak on DSC curve might be the signal of melting. Form E was speculated as an anisole solvate.

### EXAMPLE 6A: Preparation of Compound 1 Form F

Amorphous Compound 1 (20 mg) was suspended in 0.5 mL EtOH, stirred at 50° C., to obtain F.

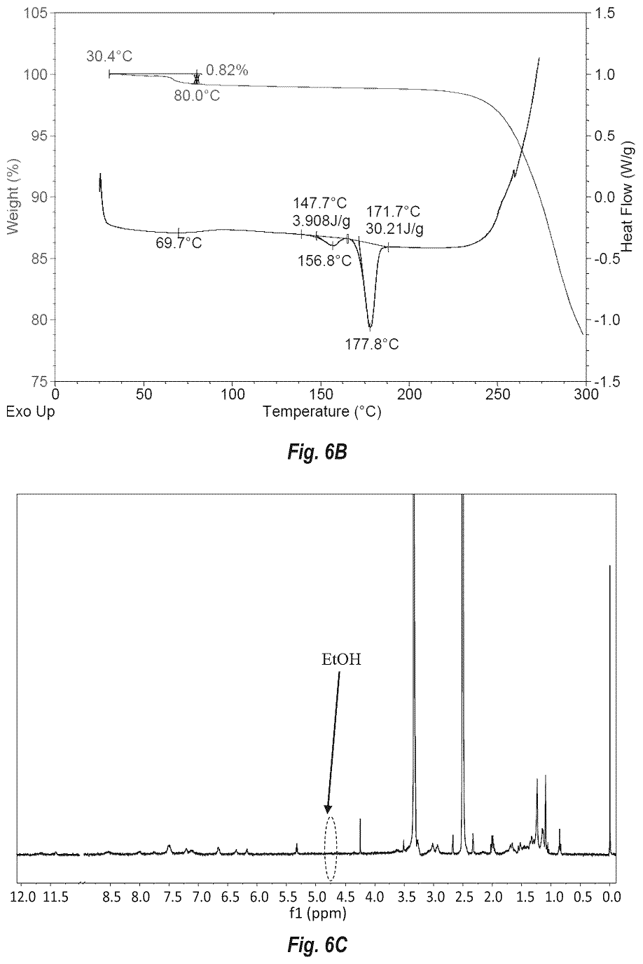

The X-ray powder diffraction (XRPD) pattern was used to characterize the obtained Form F, which showed that Form F was in a crystalline form, see FIG. 6A. The characteristic peaks and percent peak intensities obtained from the XRPD analysis are listed in Table 6A.

TABLE 6A
XRPD pattern of Compound 1 Form F

| Pos. [°2θ] | d-spacing [Å] | Rel. Int. [%] |
|---|---|---|
| 6.84 | 12.92 | 100.00 |
| 8.66 | 10.21 | 3.90 |
| 10.50 | 8.43 | 17.22 |
| 10.85 | 8.16 | 4.13 |
| 12.55 | 7.05 | 40.33 |
| 13.26 | 6.68 | 6.78 |
| 13.66 | 6.48 | 40.53 |
| 14.02 | 6.31 | 39.46 |
| 14.56 | 6.08 | 7.50 |
| 14.81 | 5.98 | 11.14 |
| 16.34 | 5.43 | 28.89 |
| 16.83 | 5.27 | 21.43 |
| 17.33 | 5.12 | 3.01 |
| 18.35 | 4.84 | 10.27 |
| 19.05 | 4.66 | 18.00 |
| 19.41 | 4.57 | 30.18 |
| 20.35 | 4.36 | 7.25 |
| 20.68 | 4.30 | 23.86 |
| 21.07 | 4.22 | 2.05 |
| 21.68 | 4.10 | 3.97 |
| 22.26 | 3.99 | 8.26 |
| 22.73 | 3.91 | 4.02 |
| 23.10 | 3.85 | 3.15 |
| 23.73 | 3.75 | 14.37 |
| 24.39 | 3.65 | 15.77 |
| 24.89 | 3.58 | 1.93 |
| 25.69 | 3.47 | 5.86 |
| 26.55 | 3.36 | 2.66 |
| 26.99 | 3.30 | 7.78 |
| 27.48 | 3.25 | 2.08 |
| 28.22 | 3.16 | 1.76 |
| 29.64 | 3.01 | 2.68 |

TGA/DSC curve showed that, a weight loss of 0.8% up to 80° C., one broad peak around 69.7° C. and two endothermic peaks at 156.8° C. and 177.8° C. (peak) before decomposition (FIG. 6B). In the ¹H NMR spectrum, no signal of EtOH was detected (FIG. 6C). Results of the heating experiment showed that no form change was observed when heating Form F to 80° C., and after heating Form F to 150° C. and 165° C., diffraction peaks of Form B were detected. According to the results of VT-XRPD, no form change was observed after heating Form F to 100° C. and cooling back to 30° C. in N2, which indicates that Form F was an anhydrate. The broad endotherm observed in DSC at 69.7° C. was speculated to be caused by loss of residual solvent or moisture, the endotherm at 156.8° C. was possibly related to form conversion at high temperature.

### EXAMPLE 7A: Preparation of Compound 1 Form G (Form G)

Amorphous Compound 1 (20 mg) was suspended in MTBE. The suspension was subjected to slurry at RT by stirring for 1˜7 days, to obtain Form G.

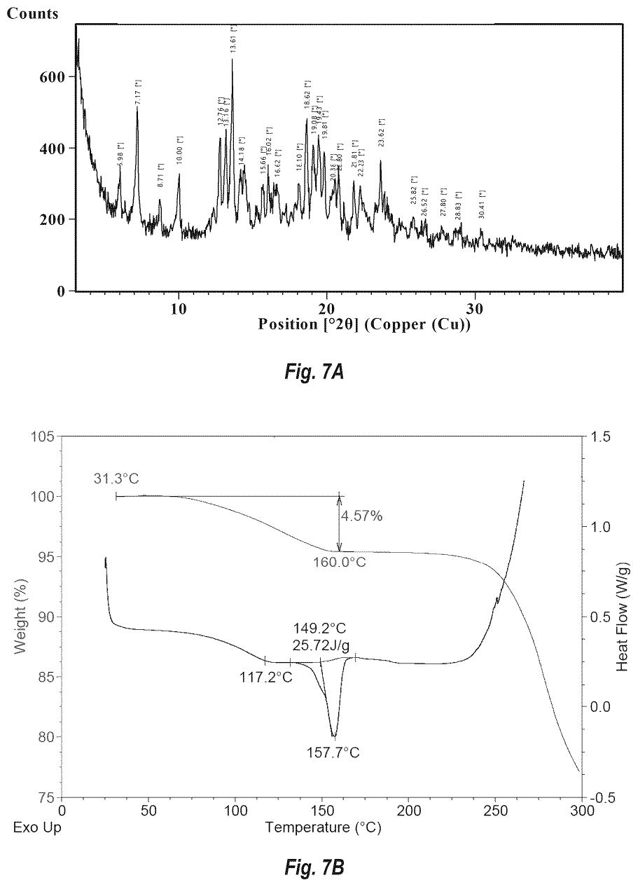

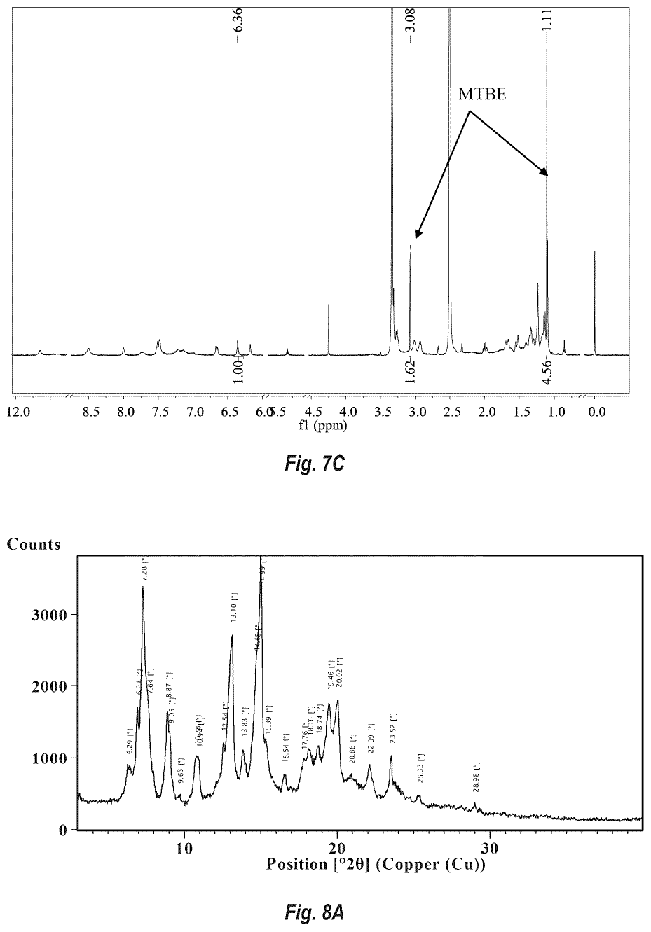

The X-ray powder diffraction (XRPD) pattern was used to characterize the obtained Form G, which showed that Form G was in a crystalline form, see FIG. 7A. The characteristic peaks and percent peak intensities obtained from the XRPD analysis are listed in Table 7A.

TABLE 7A
XRPD pattern of Compound 1 Form G

| Pos. [°2θ] | d-spacing [A] | Rel. Int. [%] |
|---|---|---|
| 5.98 | 14.78 | 35.87 |
| 7.17 | 12.33 | 69.66 |
| 8.71 | 10.15 | 24.51 |
| 10.00 | 8.84 | 36.98 |
| 12.76 | 6.94 | 58.14 |
| 13.16 | 6.73 | 57.59 |
| 13.61 | 6.51 | 100.00 |
| 14.18 | 6.25 | 36.45 |
| 15.66 | 5.66 | 31.51 |
| 16.02 | 5.53 | 41.86 |
| 16.62 | 5.33 | 29.84 |
| 18.10 | 4.90 | 33.05 |
| 18.62 | 4.76 | 67.01 |
| 19.08 | 4.65 | 53.52 |
| 19.43 | 4.57 | 58.83 |
| 19.81 | 4.48 | 50.65 |
| 20.38 | 4.36 | 27.44 |
| 20.80 | 4.27 | 33.06 |
| 21.81 | 4.08 | 34.69 |
| 22.23 | 4.00 | 29.86 |
| 23.62 | 3.77 | 46.42 |
| 25.82 | 3.45 | 14.89 |
| 26.52 | 3.36 | 8.36 |
| 27.80 | 3.21 | 8.42 |
| 28.83 | 3.10 | 6.55 |
| 30.41 | 2.94 | 7.11 |

TGA/DSC results showed that a weight loss of 4.6% up to 160° C., one weak endothermic peak at 117.2° C. and one strong endothermic peak 157.7° C. (peak) before decomposition were observed (FIG. 7B). According to the integration of ¹H NMR result (FIG. 7C), the theoretical weight of MTBE was calculated as 5.1%. XRPD overlay before and after heating showed after heating experiments, an obvious decrease of crystallinity was observed. Form G was speculated as an MTBE solvate.

### EXAMPLE 8A: Preparation of Compound 1 Form H (Form H)

Amorphous Compound 1 (20 mg) was suspended in ACN. The suspension was subjected to slurry at RT by stirring for 1˜7 days, to obtain Form H.

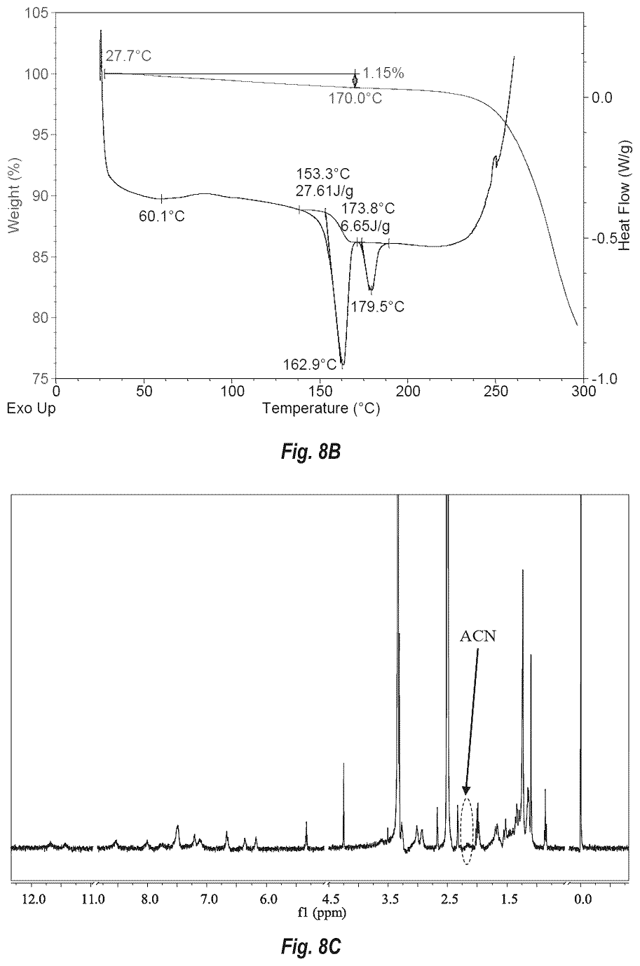

The X-ray powder diffraction (XRPD) pattern was used to characterize the obtained Form H, which showed that Form H was in a crystalline form, see FIG. 8A. The characteristic peaks and percent peak intensities obtained from the XRPD analysis are listed in Table 8A.

TABLE 8A
XRPD pattern of Compound 1 Form H

| Pos. [°2θ] | d-spacing [Å] | Rel. Int. [%] |
|---|---|---|
| 6.29 | 14.05 | 14.03 |
| 6.91 | 12.79 | 37.76 |
| 7.28 | 12.14 | 86.45 |
| 7.64 | 11.57 | 38.25 |
| 8.87 | 9.96 | 36.86 |
| 9.05 | 9.78 | 26.74 |
| 9.63 | 9.18 | 2.53 |
| 10.78 | 8.21 | 18.31 |
| 10.94 | 8.09 | 17.12 |
| 12.54 | 7.06 | 23.74 |
| 13.10 | 6.76 | 67.22 |
| 13.83 | 6.40 | 21.51 |
| 14.68 | 6.03 | 55.92 |
| 14.99 | 5.91 | 100.00 |
| 15.39 | 5.76 | 22.45 |
| 16.54 | 5.36 | 11.39 |
| 17.76 | 4.99 | 16.90 |
| 18.16 | 4.89 | 22.35 |
| 18.74 | 4.74 | 23.28 |
| 19.46 | 4.56 | 40.81 |
| 20.02 | 4.44 | 41.97 |
| 20.88 | 4.26 | 11.42 |
| 22.09 | 4.02 | 15.85 |
| 23.52 | 3.78 | 20.04 |
| 25.33 | 3.52 | 4.14 |
| 28.98 | 3.08 | 2.57 |

TGA/DSC curve showed, weight loss of 1.2% up to 170° C. and three endothermic peaks at 60.1° C., 162.9° C. and 179.5° C. (peak) before decomposition were detected (FIG. 8B). No signal of ACN was detected in the ¹H NMR result (FIG. 8C), which indicated Form H might be an anhydrate/hydrate.

### EXAMPLE 9A: Preparation of Compound 1 Form I (Form I)

Amorphous Compound 1 (20 mg) was suspended in 0.5 mL IPA, stirred at 50° C., to obtain Form I.

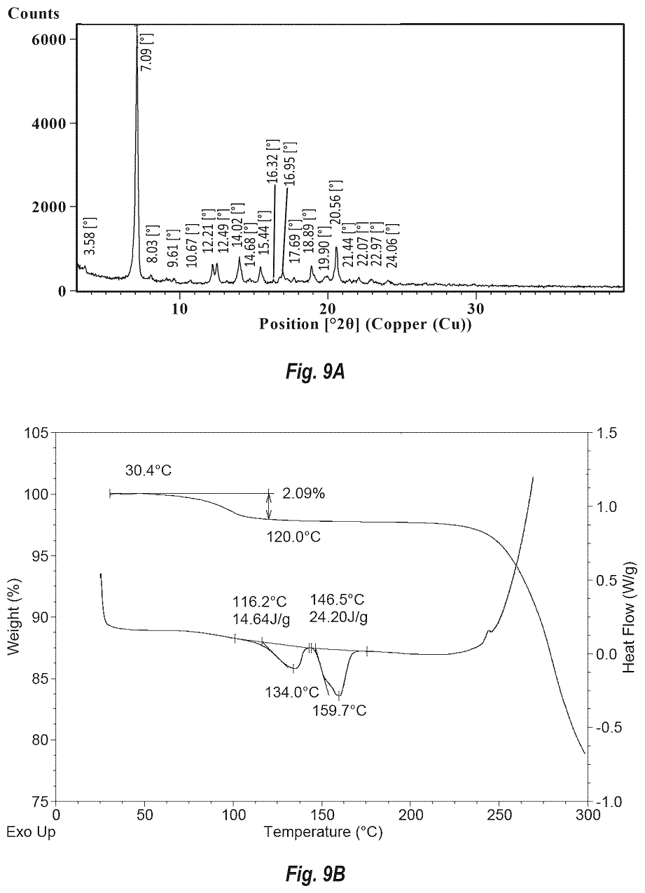

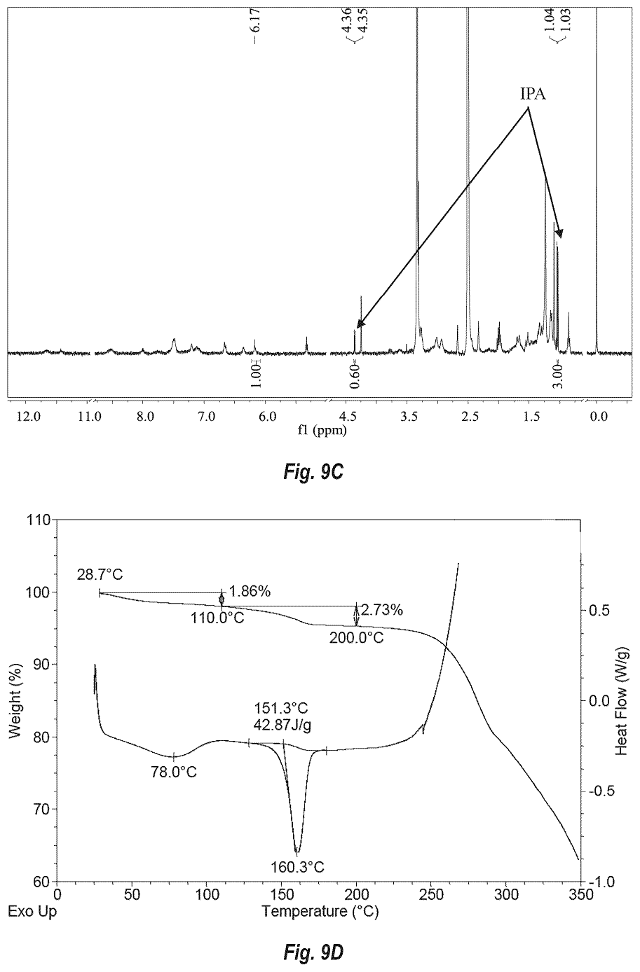

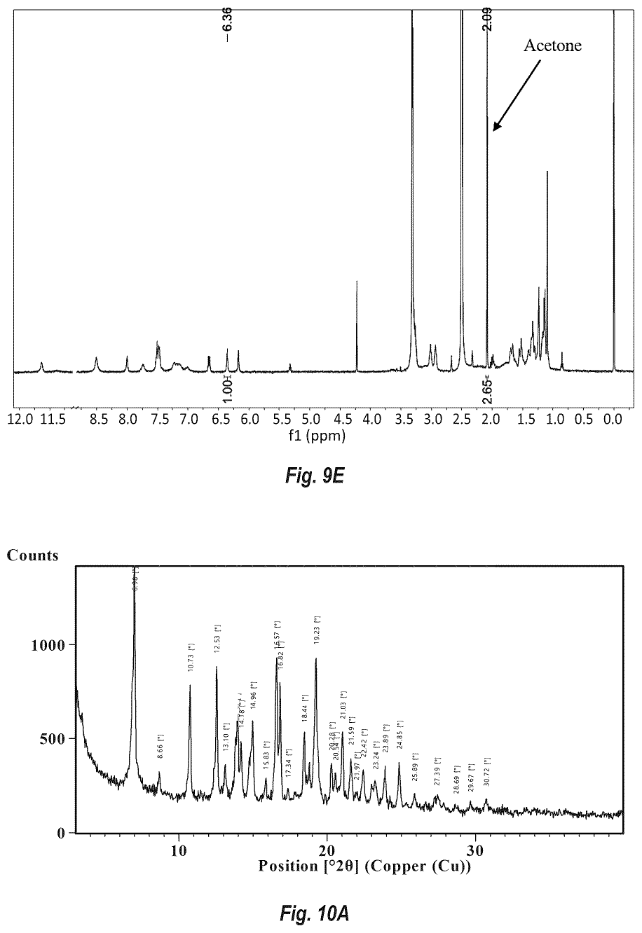

The X-ray powder diffraction (XRPD) pattern was used to characterize the obtained Form I, which showed that Form I was in a crystalline form, see FIG. 9A.

| Pos. [°2θ] | d-spacing [Å] | Rel. Int. [%] |
|---|---|---|
| 3.58 | 24.68 | 4.38 |
| 7.09 | 12.47 | 100.00 |
| 8.03 | 11.01 | 2.57 |
| 9.61 | 9.20 | 1.69 |
| 10.67 | 8.29 | 0.94 |
| 12.21 | 7.25 | 7.50 |
| 12.49 | 7.09 | 8.01 |
| 14.02 | 6.32 | 10.43 |
| 14.68 | 6.04 | 1.56 |
| 15.44 | 5.74 | 6.62 |
| 16.32 | 5.43 | 1.69 |
| 16.95 | 5.23 | 4.71 |
| 17.69 | 5.01 | 2.53 |
| 18.89 | 4.70 | 7.02 |
| 19.90 | 4.46 | 3.08 |
| 20.56 | 4.32 | 14.49 |
| 21.44 | 4.14 | 2.06 |
| 22.07 | 4.03 | 2.72 |
| 22.97 | 3.87 | 1.97 |
| 24.06 | 3.70 | 1.61 |

TGA/DSC curves showed a weight loss of 2.1% up to 120° C. and two endothermic peaks at 134.0° C. and 159.7° C. before decomposition were detected (FIG. 9B). In ¹H NMR spectrum (FIG. 9C), peaks of IPA were observed and the content was calculated as 3.2%. XRPD comparison displayed that in the heating experiment, an obvious decrease of crystallinity was observed for Form I. Form I was speculated as an IPA solvate.

### EXAMPLE 9B: Preparation of Compound 1 Form I

Form I from slow evaporation in acetone showed the same XRPD pattern with Form I in Example 9A. Two steps of TGA weight loss (1.9% up to 110° C. and 2.7% from 110° C. to 200° C., see FIG. 9D), two endothermic peaks at 78.0° C. and 160.3° C. before decomposition in DSC thermogram were observed. ¹H NMR (FIG. 9E) results displayed the content of acetone in the sample was determined as 2.8%.

According to the results of heating experiments, no form change was observed after heating Form I from acetone to 130° C. but an amorphous sample was observed when the heating temperature reached 180° C. Combined with TGA and ¹H NMR data, the first step of TGA weight loss might be the de-sorption of volatile components, while the second step of weight loss was possibly due to loss of acetone, which led to the transformation to amorphous phase. Thus, Form I from acetone was speculated as an acetone solvate.

Since different solvates were in the same XRPD pattern as Form I, it was speculated that isomorphism occurred during the formation of Form I.

### EXAMPLE 10A: Preparation of Compound 1 Form J (Form J)

Amorphous Compound 1 (20 mg) was suspended in 2-MeTHF/n-heptane (1:1, v/v). The suspension was subjected to slurry at RT by stirring for 1˜7 days, to obtain Form J.

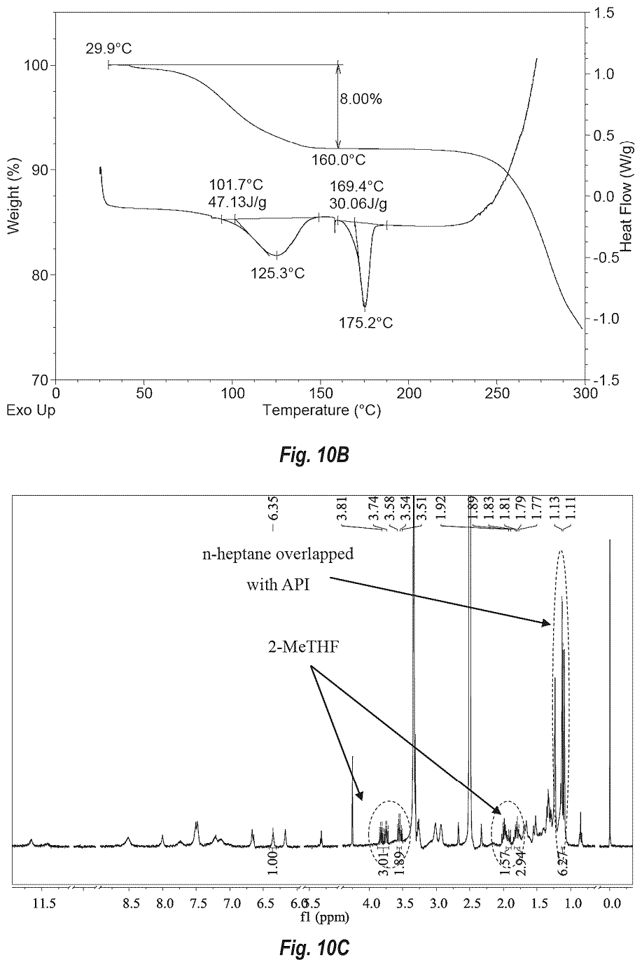

The X-ray powder diffraction (XRPD) pattern was used to characterize the obtained Form J, which showed that Form J was in a crystalline form, see FIG. 10A. The characteristic peaks and percent peak intensities obtained from the XRPD analysis are listed in Table 10A.

TABLE 10A
XRPD pattern of Compound 1 Form J

| Pos. [°2θ] | d-spacing [Å] | Rel. Int. [%] |
|---|---|---|
| 6.98 | 12.67 | 100.00 |
| 8.66 | 10.21 | 10.03 |
| 10.73 | 8.24 | 51.79 |
| 12.53 | 7.06 | 61.11 |
| 13.10 | 6.76 | 16.09 |
| 13.90 | 6.37 | 30.35 |
| 14.18 | 6.25 | 27.31 |
| 14.96 | 5.92 | 36.10 |
| 15.83 | 5.60 | 8.80 |
| 16.57 | 5.35 | 64.29 |
| 16.82 | 5.27 | 55.14 |
| 17.34 | 5.11 | 5.84 |
| 18.44 | 4.81 | 31.33 |
| 19.23 | 4.62 | 67.51 |
| 20.28 | 4.38 | 19.13 |
| 20.54 | 4.32 | 14.82 |
| 21.03 | 4.23 | 33.70 |
| 21.59 | 4.12 | 22.00 |
| 21.97 | 4.05 | 6.20 |
| 22.42 | 3.97 | 16.54 |
| 23.24 | 3.83 | 11.51 |
| 23.89 | 3.72 | 19.15 |
| 24.85 | 3.58 | 21.30 |
| 25.89 | 3.44 | 6.76 |
| 27.39 | 3.26 | 5.43 |
| 28.69 | 3.11 | 2.02 |
| 29.67 | 3.01 | 3.21 |

TGA/DSC curve showed a weight loss of 8.0% up to 160° C. and two endothermic peaks at 125.3° C. and 175.2° C. (peak) before decomposition were detected (FIG. 10B). ¹H NMR (FIG. 10C) result showed a signal of 2-MeTHF and n-heptane were observed in Form J (theoretical weight loss: ˜10.2%). XRPD overlay illustrated that after heating to 150° C. and cooling back to RT, Form J converted to Form B. Based on the TGA, ¹H NMR and heating experiment data, Form J was speculated as a 2-MeTHF solvate.

In addition, Form J was heated to 130° C. and then being isothermal at 130° C. for 30 min, and then was cooled down to RT. XRPD results shown that Form B of low crystallinity was obtained.

### EXAMPLE 11A: Preparation of Compound 1 Form K (Form K)

Amorphous Compound 1 (20 mg) was suspended in methyl acetate. The suspension was subjected to slurry at RT by stirring for 1˜7 days, to obtain Form K.

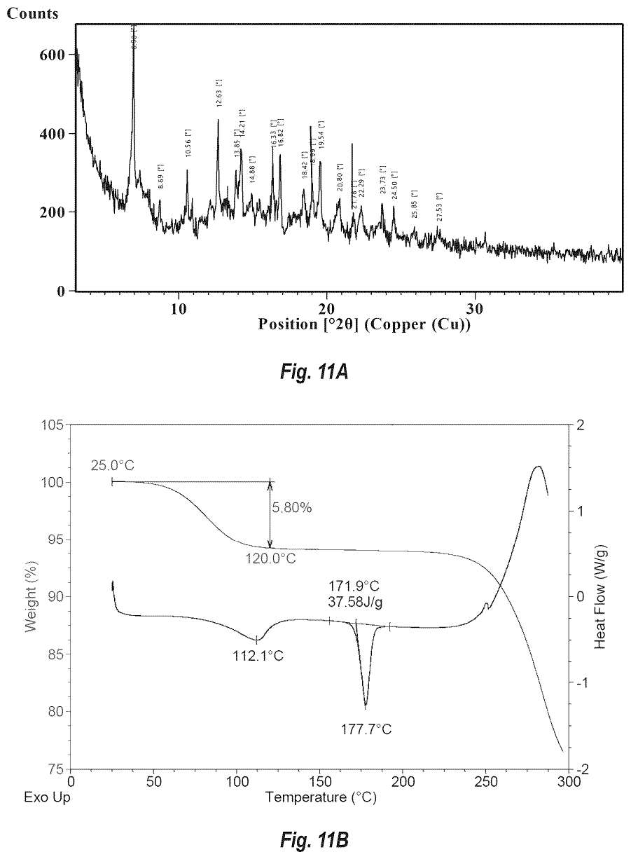

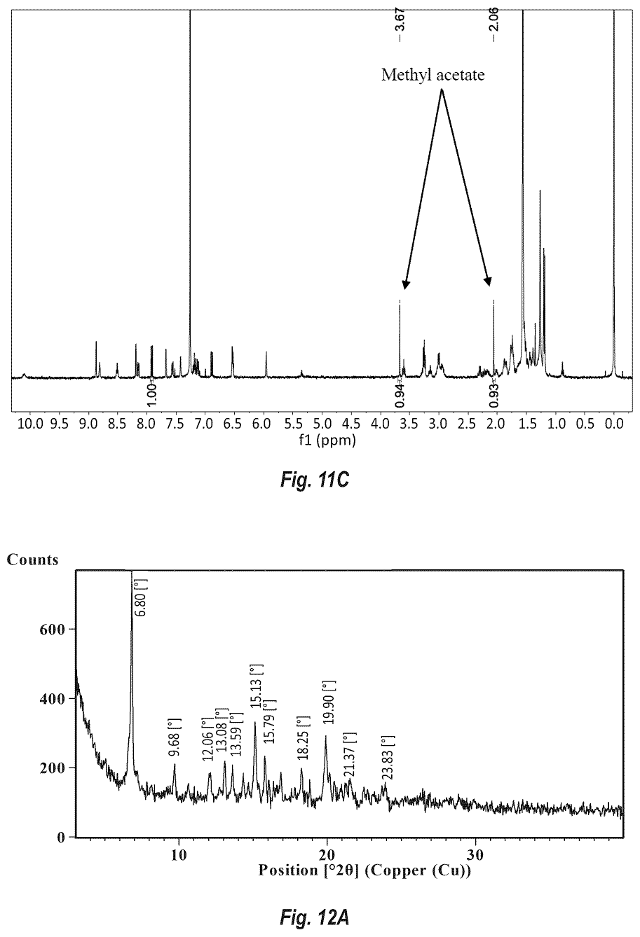

The X-ray powder diffraction (XRPD) pattern was used to characterize the obtained Form K, which showed that Form K was in a crystalline form, see FIG. 11A. The characteristic peaks and percent peak intensities obtained from the XRPD analysis are listed in Table 11A.

TABLE 11A
XRPD pattern of Compound 1 Form K [%]

| Pos. [°2θ] | d-spacing [Å] | Rel. Int. [%] |
|---|---|---|
| 6.90 | 12.81 | 100.00 |
| 8.69 | 10.17 | 13.04 |
| 10.56 | 8.38 | 36.54 |
| 12.63 | 7.01 | 68.65 |
| 13.85 | 6.39 | 38.66 |
| 14.21 | 6.23 | 51.09 |
| 14.88 | 5.95 | 22.63 |
| 16.33 | 5.43 | 45.89 |
| 16.82 | 5.27 | 47.13 |
| 18.42 | 4.82 | 27.88 |
| 18.99 | 4.67 | 36.24 |
| 19.54 | 4.54 | 47.49 |
| 20.80 | 4.27 | 21.69 |
| 21.78 | 4.08 | 11.61 |
| 22.29 | 3.99 | 19.17 |
| 23.73 | 3.75 | 21.88 |
| 24.50 | 3.63 | 19.06 |
| 25.85 | 3.45 | 8.16 |
| 27.53 | 3.24 | 7.32 |

GA/DSC showed that there was a weight loss of 5.8% up to 120° C. and two endothermic peaks at 112.1° C. and 177.7° C. (peak) before decomposition (FIG. 11B). In ¹H NMR (FIG. 11C), the signal of methyl acetate was observed with a theoretical weight loss ˜2.5%.

XRPD overlay of the heating experiment showed Form B of weak crystallinity was observed after heating Form K to 120° C. As XRPD overlay showed, after storage at RT in a closed HPLC vial for ˜5 weeks, Form K converted to Form B of low crystallinity. Form K was speculated as a methyl acetate solvate.

### EXAMPLE 11B: Preparation of Compound 1 Form K

Compound 1 (8.0 g) was added into methyl acetate (100 mL) then was heated to 50° C. for 2 hours. The mixture was cooled down to RT and stirred for 16 hours. The mixture was filtrated, washed with methyl acetate and dried over reduced pressure to give the product (7.1 g).

### EXAMPLE 12A: Preparation of Compound 1 Form L (Form L)

Amorphous Compound 1 (20 mg) was suspended in 0.5 mL acetone/n-heptane (1:1, v/v), stirred at 50° C., to obtain Form L.

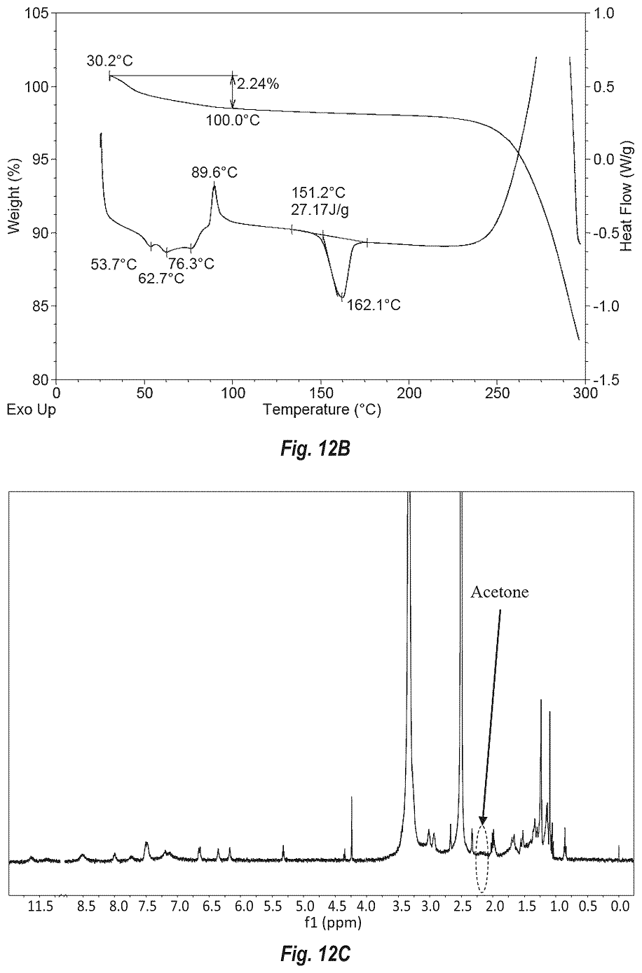

The X-ray powder diffraction (XRPD) pattern was used to characterize the obtained Form L, which showed that Form L was in a crystalline form, see FIG. 12A. The characteristic peaks and percent peak intensities obtained from the XRPD analysis are listed in Table 12A.

TABLE 12A
XRPD pattern of Compound 1 Form L

| Pos. [°2θ] | d-spacing [Å] | Rel. Int. [%] |
|---|---|---|
| 6.80 | 13.00 | 100.00 |
| 9.68 | 9.14 | 12.98 |
| 12.06 | 7.34 | 10.20 |
| 13.08 | 6.77 | 16.45 |
| 13.59 | 6.52 | 12.89 |
| 15.13 | 5.86 | 34.96 |
| 15.79 | 5.61 | 19.25 |
| 18.25 | 4.86 | 13.64 |
| 19.90 | 4.46 | 29.09 |
| 21.37 | 4.16 | 6.13 |
| 23.83 | 3.73 | 5.23 |

TGA/DSC curves showed that: weight loss of 2.2% up to 100° C. was observed in TGA plot; and, multiple signals, including four endothermic peaks at 53.7° C., 62.7° C., 76.3° C. and 162.1° C. (peak), one exothermal peak at 89.6° C. before decomposition were detected in the DSC curve (FIG. 12B). Based on ¹H NMR spectrum (FIG. 12C), no peak of acetone was observed. Thus, Form L was possibly an anhydrate/hydrate.

Form L converted to another form when being in as a wet sample, and Form L converted to Form I by storage at RT. Thus, Form L was speculated as a metastable anhydrate/hydrate which could be de-solvated from the wet cake from the solvent system.

### EXAMPLE 13A: Preparation of Compound 1 Form M (Form M)

Amorphous Compound 1 (20 mg) was suspended in CHCl3/n-heptane (1:1, v/v). The suspension was subjected to temperature cycling from 50° C. to 5° C., to obtain Form M.

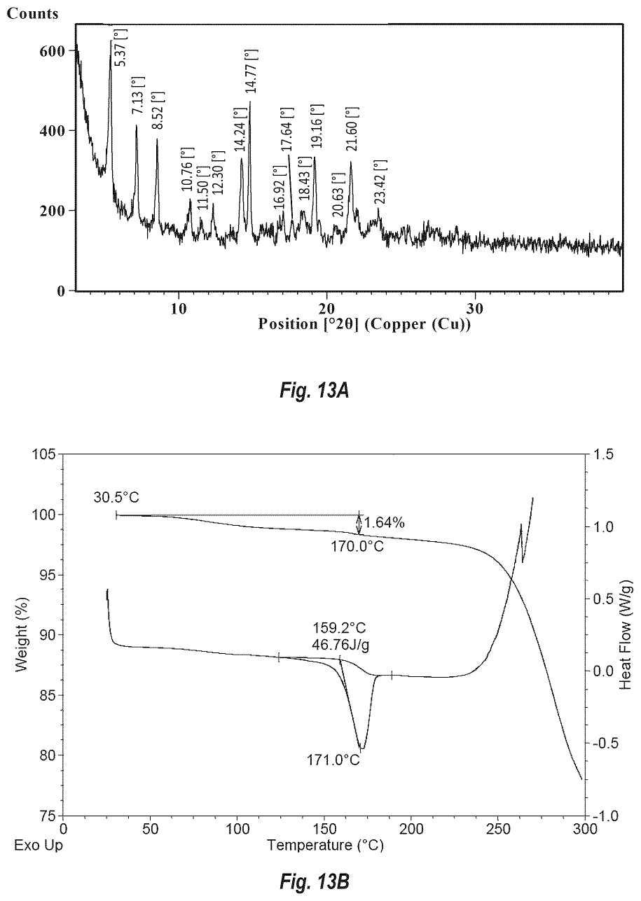

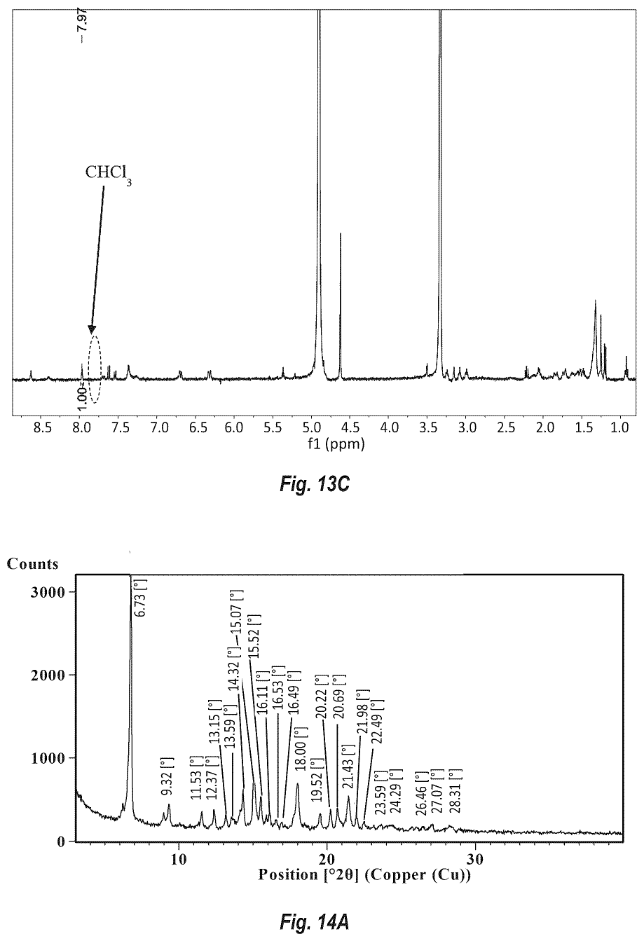

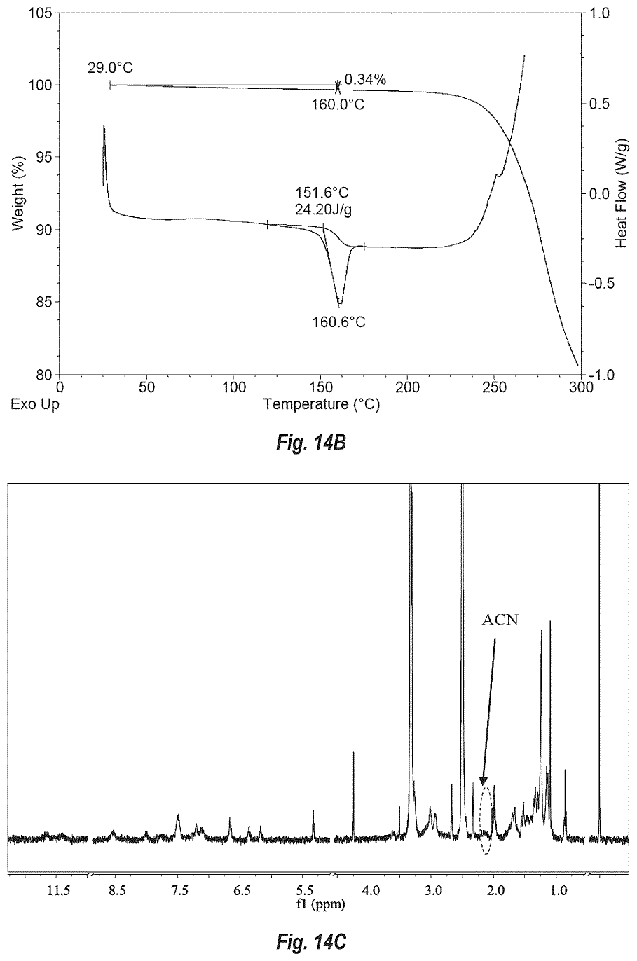

The X-ray powder diffraction (XRPD) pattern was used to characterize the obtained Form M, which showed that Form M was in a crystalline form, see FIG. 13A. The characteristic peaks and percent peak intensities obtained from the XRPD analysis are listed in Table 13A.

TABLE 13A
XRPD pattern of Compound 1 Form M

| Pos. [°2θ] | d-spacing [Å] | Rel. Int. [%] |
|---|---|---|
| 5.37 | 16.47 | 100.00 |
| 7.13 | 12.39 | 63.54 |
| 8.52 | 10.38 | 59.43 |
| 10.76 | 8.23 | 24.08 |
| 11.50 | 7.69 | 10.18 |
| 12.30 | 7.20 | 21.26 |
| 14.24 | 6.22 | 53.69 |
| 14.77 | 6.00 | 89.84 |
| 16.92 | 5.24 | 10.90 |
| 17.64 | 5.03 | 12.50 |
| 18.43 | 4.81 | 18.15 |
| 19.16 | 4.63 | 54.84 |
| 20.63 | 4.31 | 7.01 |
| 21.60 | 4.11 | 51.33 |
| 23.42 | 3.80 | 13.32 |

TGA/DSC curve showed a weight loss of 1.6% up to 170° C. and one endothermic peak at 171.0° C. (peak) before decomposition (FIG. 13B). Based on ¹H NMR result, no obvious signal of CHCl3 was observed (FIG. 13C). VT-XPRD results showed that no form change was observed after heating Form M to 120° C. and cooling back to 30° C. in N2, which indicates that From M was an anhydrate.

### EXAMPLE 14A: Preparation of Compound 1 Form N (Form N)

Amorphous Compound 1 (20 mg) was suspended in 0.5 mL ACN, stirred at 50° C., to obtain Form N.

The X-ray powder diffraction (XRPD) pattern was used to characterize the obtained Form N, which showed that Form N was in a crystalline form, see FIG. 14A. The characteristic peaks and percent peak intensities obtained from the XRPD analysis are listed in Table 14A.

TABLE 14A
XRPD pattern of Compound 1 Form N

| Pos. [°2θ] | d-spacing [Å] | Rel. Int. [%] |
|---|---|---|
| 6.73 | 13.13 | 100.00 |
| 9.32 | 9.49 | 8.75 |
| 11.53 | 7.67 | 6.33 |
| 12.37 | 7.16 | 7.10 |
| 13.15 | 6.73 | 5.35 |
| 13.59 | 6.52 | 3.45 |
| 14.32 | 6.19 | 15.35 |
| 15.07 | 5.88 | 23.16 |
| 15.52 | 5.71 | 12.47 |
| 16.11 | 5.50 | 6.41 |
| 16.53 | 5.36 | 3.46 |
| 16.94 | 5.23 | 2.46 |
| 18.00 | 4.93 | 18.18 |
| 19.52 | 4.55 | 6.27 |
| 20.22 | 4.39 | 8.03 |
| 20.69 | 4.29 | 8.63 |
| 21.43 | 4.15 | 13.04 |
| 21.98 | 4.04 | 4.63 |
| 22.49 | 3.95 | 3.42 |
| 23.59 | 3.77 | 1.73 |
| 24.29 | 3.66 | 1.84 |
| 26.46 | 3.37 | 1.24 |
| 27.07 | 3.29 | 2.28 |
| 28.31 | 3.15 | 1.80 |

TGA/DSC curve showed that a weight loss of 0.3% up to 160° C. and one endothermic peak at 160.6° C. (peak) before decomposition was observed (FIG. 14B). ¹H NMR results showed that there was no signal of ACN (FIG. 14C). Combined with the TGA and ¹H NMR data, Form N was speculated to be an anhydrate.

### EXAMPLE 15A: Preparation of Compound 1 Form O (Form O)

Amorphous Compound 1 (20 mg) was suspended in 0.5 mL toluene at 50° C., stirred at 50° C., to obtain Form O.

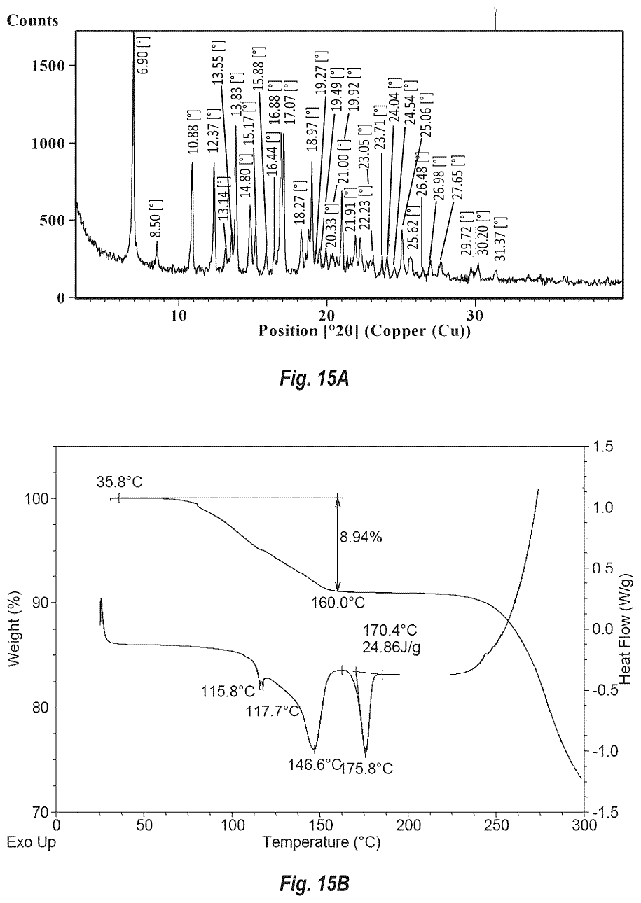

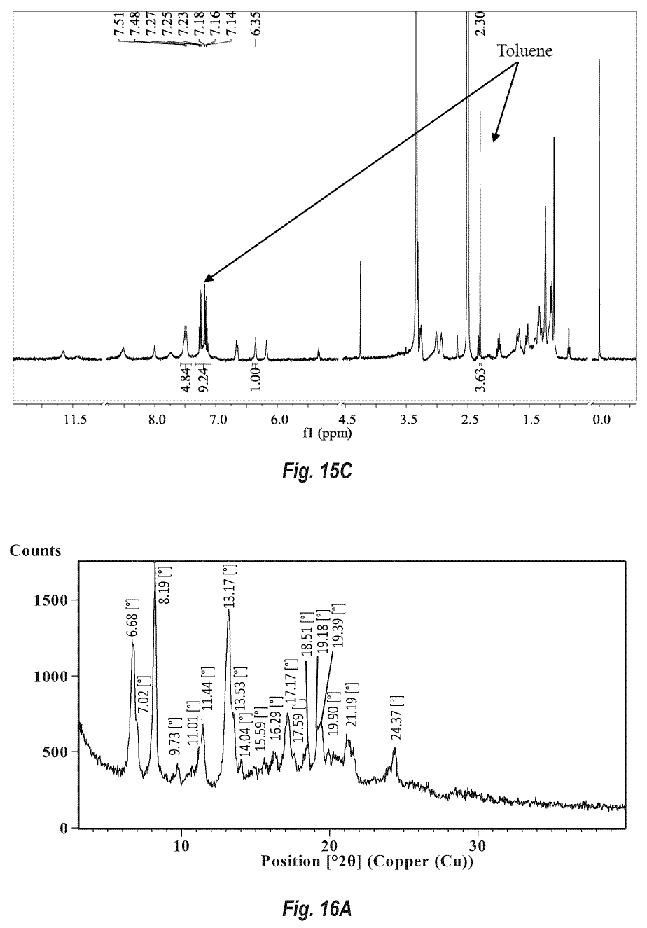

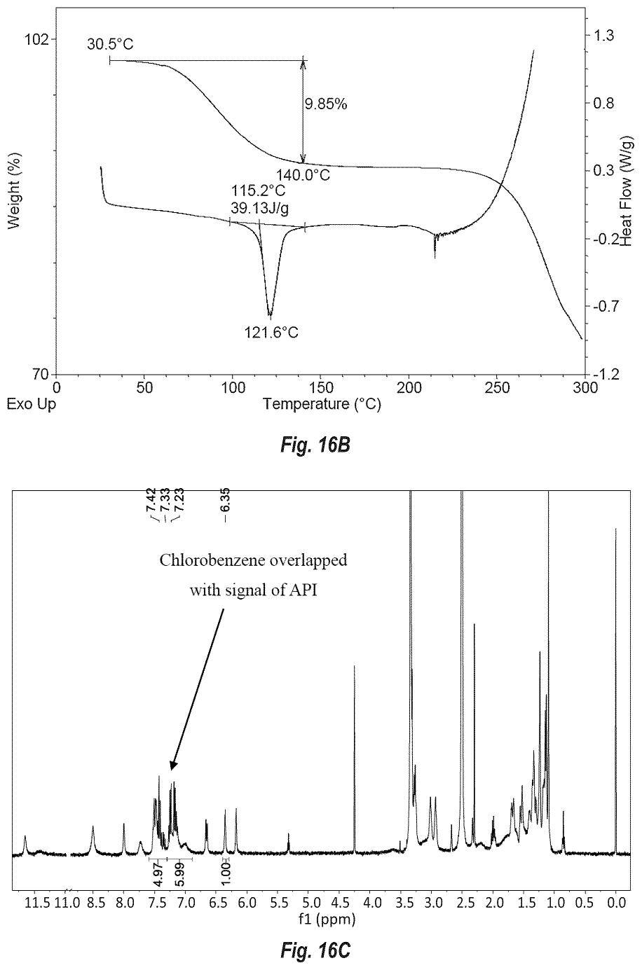

The X-ray powder diffraction (XRPD) pattern was used to characterize the obtained Form O, which showed that Form O was in a crystalline form, see FIG. 15A. The characteristic peaks and percent peak intensities obtained from the XRPD analysis are listed in Table 15A.

TABLE 15A
XRPD pattern of Compound 1 Form O

| Pos. [°2θ] | d-spacing [Å] | Rel. Int. [%] |
|---|---|---|
| 6.90 | 12.81 | 100.00 |
| 8.50 | 10.41 | 9.61 |
| 10.88 | 8.13 | 53.19 |
| 12.37 | 7.16 | 52.85 |
| 13.14 | 6.74 | 11.30 |
| 13.55 | 6.54 | 21.25 |
| 13.83 | 6.40 | 70.34 |
| 14.80 | 5.99 | 32.57 |
| 15.17 | 5.84 | 22.34 |
| 15.88 | 5.58 | 13.21 |
| 16.44 | 5.39 | 8.86 |
| 16.88 | 5.25 | 68.94 |
| 17.07 | 5.20 | 68.37 |
| 18.27 | 4.86 | 19.15 |
| 18.97 | 4.68 | 55.62 |
| 19.27 | 4.61 | 13.03 |
| 19.49 | 4.55 | 12.56 |
| 19.92 | 4.46 | 11.93 |
| 20.33 | 4.37 | 8.46 |
| 21.00 | 4.23 | 40.76 |
| 21.91 | 4.06 | 19.76 |
| 22.23 | 4.00 | 18.01 |
| 23.05 | 3.86 | 6.26 |
| 23.71 | 3.75 | 9.70 |
| 24.04 | 3.70 | 9.97 |
| 24.54 | 3.63 | 4.45 |
| 25.06 | 3.55 | 22.88 |
| 25.62 | 3.48 | 9.59 |
| 26.48 | 3.37 | 4.21 |
| 26.98 | 3.31 | 8.87 |
| 27.65 | 3.23 | 7.28 |
| 29.72 | 3.01 | 6.34 |

TGA/DSC curve showed that a weight loss of 8.9% up to 160° C. and four endothermic peaks at 115.8° C., 117.7° C., 146.6° C., 175.8° C. (peak) before decomposition were observed (FIG. 15B). In the ¹H NMR spectrum (FIG. 15C), the peak of toluene was observed and the theoretical weight loss was determined as 11.0%. The higher theoretical weight loss might be caused by inhomogeneous solvent residual. Results of the heating experiment showed that after heating to 160° C. and cooling back to RT, Form O converted to Form B. Combined with the TGA and ¹H NMR data, Type O was speculated as a toluene solvate.

### EXAMPLE 16A: Preparation of Compound 1 Form P (Form P)

Compound 1 (20 mg) was dissolved with chlorobenzene, and centrifugated. The supernatant was exposed toluene at RT, to obtain Form P.

The X-ray powder diffraction (XRPD) pattern was used to characterize the obtained Form P, which showed that Form P was in a crystalline form, see FIG. 16A. The characteristic peaks and percent peak intensities obtained from the XRPD analysis are listed in Table 16A.

TABLE 16A
XRPD pattern of Compound 1 Form P

| Pos. [°2θ] | d-spacing [Å] | Rel. Int. [%] |
|---|---|---|
| 6.68 | 13.23 | 58.51 |
| 7.02 | 12.60 | 21.55 |
| 8.19 | 10.80 | 100.00 |
| 9.73 | 9.09 | 6.04 |
| 11.01 | 8.03 | 10.11 |
| 11.44 | 7.74 | 26.79 |
| 13.17 | 6.72 | 80.77 |
| 13.53 | 6.54 | 27.66 |
| 14.04 | 6.31 | 9.60 |
| 15.59 | 5.68 | 9.22 |
| 16.29 | 5.44 | 13.17 |
| 17.17 | 5.16 | 31.50 |
| 17.59 | 5.04 | 12.69 |
| 18.51 | 4.79 | 19.35 |
| 19.18 | 4.63 | 24.97 |
| 19.39 | 4.58 | 28.13 |
| 19.90 | 4.46 | 15.46 |
| 21.19 | 4.19 | 20.11 |
| 24.37 | 3.65 | 15.17 |

TGA/DSC results displayed a weight loss of 9.9% up to 140° C. and one endothermic peak at 121.6° C. (peak) before decomposition (FIG. 16B). According to the integration of ¹H NMR result (FIG. 16C), the theoretical weight of chlorobenzene was calculated as 9.8%, which matched with the TGA weight loss. XRPD comparison showed that after storage at RT for ˜4 weeks, some of the diffraction peaks disappeared. After heating the sample to 140° C., more diffraction peaks disappeared. Combined with the TGA, ¹H NMR data and heating experiment, Form P was speculated as a chlorobenzene solvate.

### EXAMPLE 17A: Preparation of Compound 1 Form Q (Form Q)

Amorphous Compound 1 (20 mg) was subjected to solid vapor diffusion in 1,4-dioxane, at RT for 10 days, to obtain Form Q.

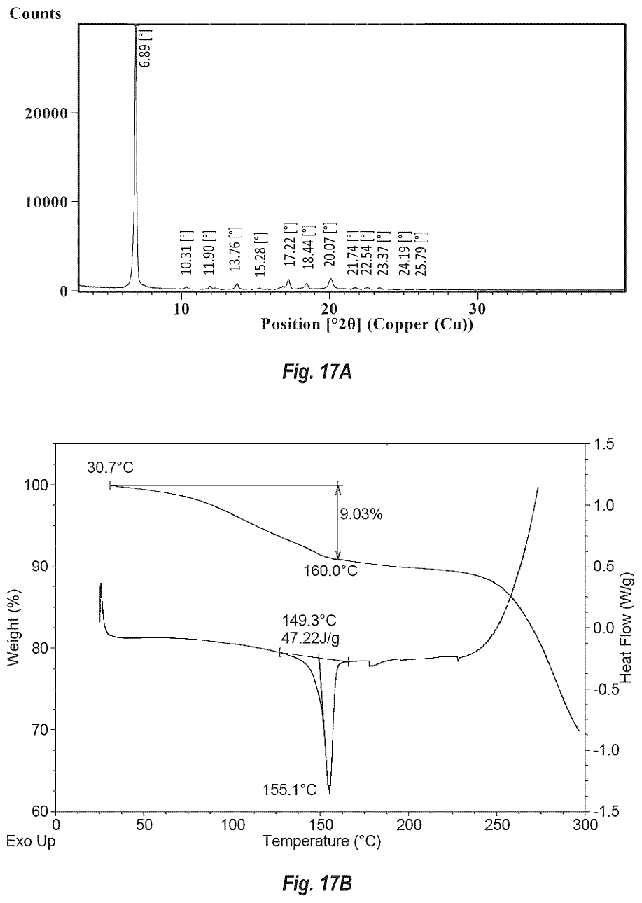

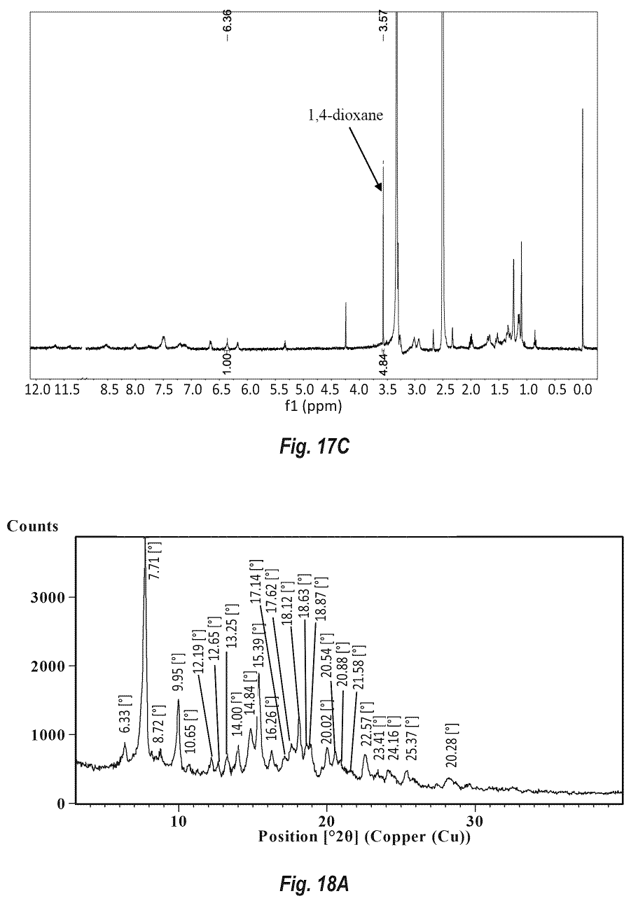

The X-ray powder diffraction (XRPD) pattern was used to characterize the obtained Form Q, which showed that Form Q was in a crystalline form, see FIG. 17A. The characteristic peaks and percent peak intensities obtained from the XRPD analysis are listed in Table 17A.

TABLE 17A
XRPD pattern of Compound 1 Form Q

| Pos. [°2θ] | d-spacing [Å] | Rel. Int. [%] |
|---|---|---|
| 6.89 | 12.83 | 100.00 |
| 10.31 | 8.58 | 0.85 |
| 11.90 | 7.44 | 1.19 |
| 13.76 | 6.44 | 2.12 |
| 15.28 | 5.80 | 0.48 |
| 17.22 | 5.15 | 3.72 |
| 18.44 | 4.81 | 2.30 |
| 20.07 | 4.42 | 4.18 |
| 21.74 | 4.09 | 0.74 |
| 22.54 | 3.94 | 0.80 |
| 23.37 | 3.81 | 0.67 |
| 24.91 | 3.57 | 0.18 |
| 25.79 | 3.45 | 0.23 |

TGA/DSC showed a weight loss of 9.0% up to 160° C. and one endothermic peak at 155.1° C. (peak) before decomposition (FIG. 17B). In the ¹H NMR spectrum (FIG. 17C), a peak of 1,4-dioxane was detected with a theoretical weight of 5.6%. The theoretical weight loss was lower than TGA weight loss, which might be caused by the solvent loss during storage.

Form Q was speculated as a 1,4-dioxane solvate.

### EXAMPLE 18A: Preparation of Compound 1 Form R (Form R)

Amorphous Compound 1 (about 100 mg) was suspended in 0.5 mL ACN, to obtain Form R.

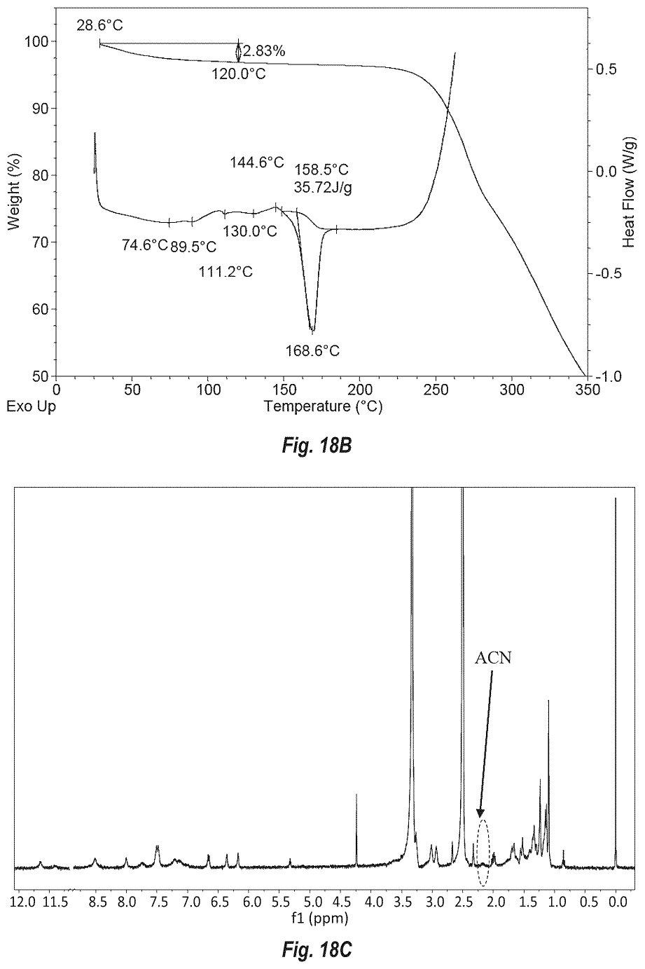

The X-ray powder diffraction (XRPD) pattern was used to characterize the obtained Form R, which showed that Form R was in a crystalline form, see FIG. 18A. The characteristic peaks and percent peak intensities obtained from the XRPD analysis are listed in Table 18A.

TABLE 18A
XRPD pattern of Compound 1 Form R

| Pos. [°2θ] | d-spacing [Å] | Rel. Int. [%] |
|---|---|---|
| 6.33 | 13.97 | 10.48 |
| 7.71 | 11.47 | 100.00 |
| 8.72 | 10.14 | 6.79 |
| 9.95 | 8.89 | 30.90 |
| 10.65 | 8.30 | 3.13 |
| 12.19 | 7.26 | 6.78 |
| 12.65 | 7.00 | 5.42 |
| 13.25 | 6.68 | 7.15 |
| 14.00 | 6.33 | 12.62 |
| 14.84 | 5.97 | 20.21 |
| 15.39 | 5.76 | 44.58 |
| 16.26 | 5.45 | 11.53 |
| 17.14 | 5.17 | 9.11 |
| 17.62 | 5.03 | 14.23 |
| 18.12 | 4.90 | 26.07 |
| 18.63 | 4.76 | 15.33 |
| 18.87 | 4.70 | 15.19 |
| 20.02 | 4.44 | 13.86 |
| 20.54 | 4.32 | 12.96 |
| 20.88 | 4.26 | 8.89 |
| 21.58 | 4.12 | 4.86 |
| 22.57 | 3.94 | 11.69 |
| 23.41 | 3.80 | 4.88 |
| 24.16 | 3.68 | 5.51 |
| 25.37 | 3.51 | 5.98 |
| 28.20 | 3.16 | 3.92 |

TGA/DSC curves showed a weight loss of 2.8% up to 120° C. and five endothermic peaks at 74.6° C., 89.5° C., 111.2° C., 130.0° C., 168.6° C. (peak), one exothermal peak at 144.6° C. before decomposition (FIG. 18B). In ¹H NMR spectrum (FIG. 18C), no signal of ACN was observed. VT-XRPD showed that: after drying Form R by N2 for about 20 mins, no form change was observed; after heating Form R to 100° C. and cooling back to 30° C. in N2, extra peaks and obvious peak shifts were observed. Considering the complex of thermal signals observed in DSC before 100° C., Form R was speculated as an anhydrate/hydrate.

### EXAMPLE 19A: Preparation of Compound 1 Form S (Form S)

Compound 1 Form R was heated to 150° C. in N2 atmosphere then cooling back to 30° C., to obtain Form S.

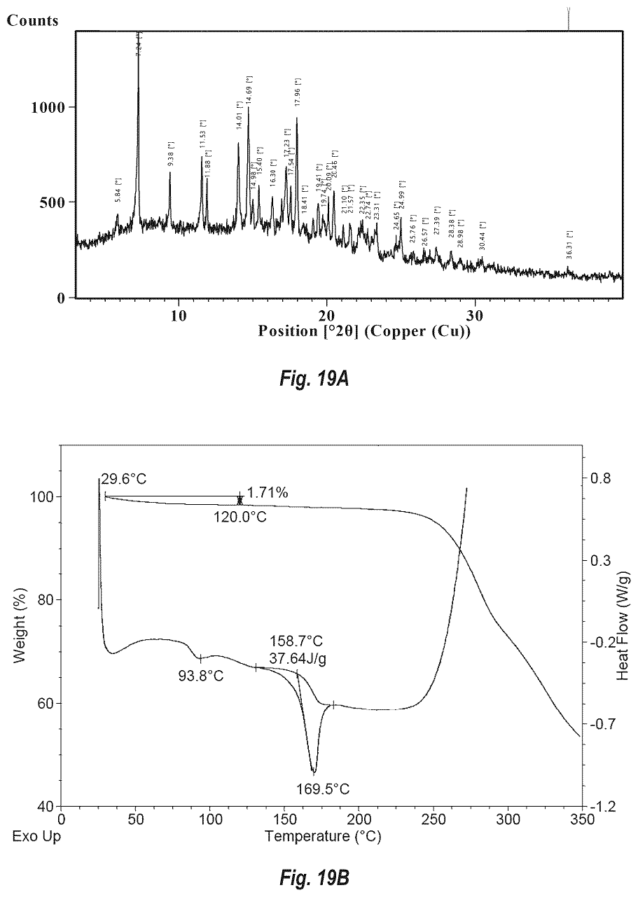

The X-ray powder diffraction (XRPD) pattern was used to characterize the obtained Form S, which showed that Form S was in a crystalline form, see FIG. 19A. The characteristic peaks and percent peak intensities obtained from the XRPD analysis are listed in Table 19A.

TABLE 19A
XRPD pattern of Compound 1 Form S

| Pos. [°2θ] | d-spacing [Å] | Rel. Int. [%] |
|---|---|---|
| 5.84 | 15.14 | 15.29 |
| 7.24 | 12.22 | 100.00 |
| 9.38 | 9.43 | 37.96 |
| 11.53 | 7.67 | 49.13 |
| 11.88 | 7.45 | 32.48 |
| 14.01 | 6.32 | 61.09 |
| 14.69 | 6.03 | 75.74 |
| 14.98 | 5.91 | 27.89 |
| 15.40 | 5.76 | 36.26 |
| 16.30 | 5.44 | 30.21 |
| 17.23 | 5.15 | 47.74 |
| 17.54 | 5.06 | 37.46 |
| 17.96 | 4.94 | 76.34 |
| 18.41 | 4.82 | 16.47 |
| 19.41 | 4.57 | 29.43 |
| 19.74 | 4.50 | 20.80 |
| 20.09 | 4.42 | 30.41 |
| 20.46 | 4.34 | 37.54 |
| 21.10 | 4.21 | 18.10 |
| 21.57 | 4.12 | 18.65 |
| 22.35 | 3.98 | 18.57 |
| 22.74 | 3.91 | 15.87 |
| 23.31 | 3.82 | 15.84 |
| 24.65 | 3.61 | 11.24 |
| 24.99 | 3.56 | 22.96 |
| 25.76 | 3.46 | 4.45 |
| 26.57 | 3.35 | 3.45 |
| 27.39 | 3.26 | 10.16 |
| 28.38 | 3.14 | 9.01 |
| 28.98 | 3.08 | 3.76 |

The TGA/DSC curves showed that a weight loss of 1.7% up to 120° C. and two endothermic peaks at 93.8° C. and 169.5° C. before decomposition was observed (FIG. 19B).

### EXAMPLE 20A: Preparation of Compound 1 Form T (Form T)

After keeping Form N under 25° C./60% RH and 40° C./75% RH for one week, and 80° C./sealed for 24 hrs, Form N converted to Form T. However, after storing for 3 days under the same conditions, Form T converted back to Form N.

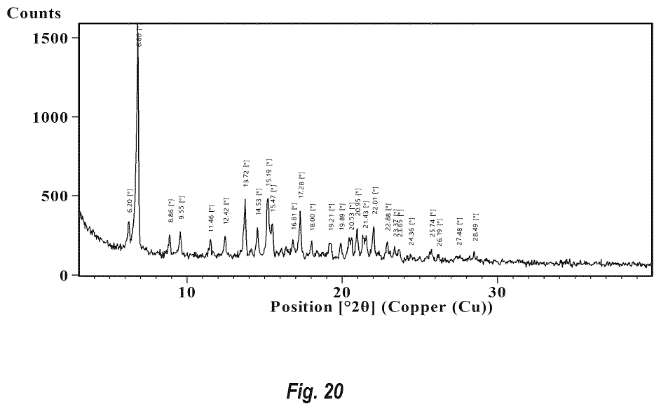

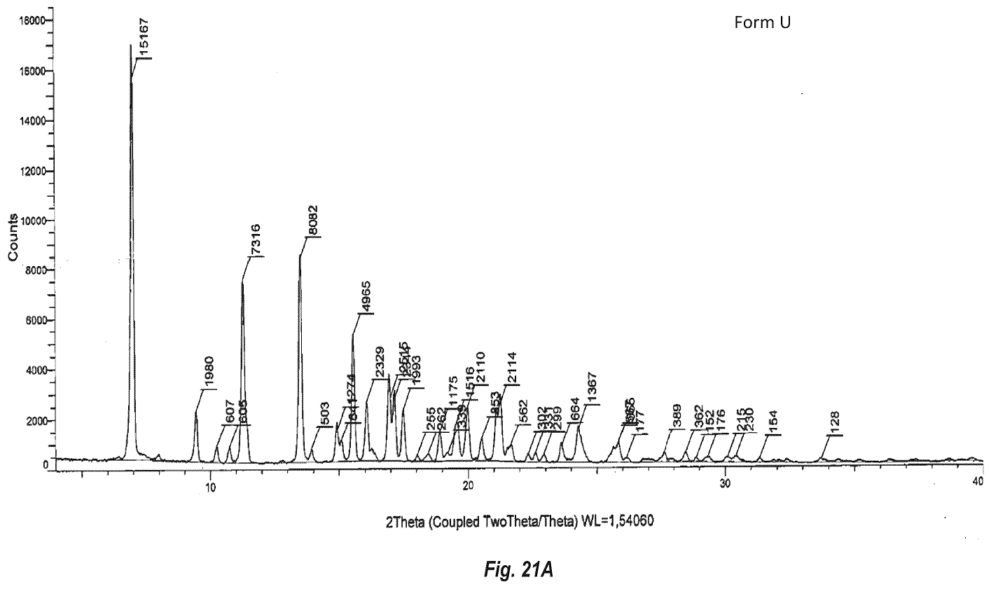

The X-ray powder diffraction (XRPD) pattern was used to characterize the obtained Form T, which showed that Form T was in a crystalline form, see FIG. 20. The characteristic peaks and percent peak intensities obtained from the XRPD analysis are listed in Table 20A.

TABLE 20A
XRPD pattern of Compound 1 Form T

| Pos. [°2θ] | d-spacing [Å] | Rel. Int. [%] |
|---|---|---|
| 6.20 | 14.25 | 10.69 |
| 6.80 | 13.00 | 100.00 |
| 8.86 | 9.98 | 8.80 |
| 9.55 | 9.26 | 10.52 |
| 11.46 | 7.72 | 5.34 |
| 12.42 | 7.13 | 8.73 |
| 13.72 | 6.45 | 25.47 |
| 14.53 | 6.10 | 13.02 |
| 15.19 | 5.83 | 25.93 |
| 15.47 | 5.73 | 14.73 |
| 16.81 | 5.28 | 6.96 |
| 17.28 | 5.13 | 20.61 |
| 18.00 | 4.93 | 6.69 |
| 19.21 | 4.62 | 6.85 |
| 19.89 | 4.46 | 7.13 |
| 20.53 | 4.33 | 7.63 |
| 20.95 | 4.24 | 13.74 |
| 21.43 | 4.15 | 8.46 |
| 22.01 | 4.04 | 14.71 |
| 22.88 | 3.89 | 7.58 |
| 23.37 | 3.81 | 5.11 |
| 23.65 | 3.76 | 4.84 |
| 24.36 | 3.65 | 1.30 |
| 25.74 | 3.46 | 5.19 |
| 26.19 | 3.40 | 1.32 |
| 27.48 | 3.25 | 1.72 |
| 28.49 | 3.13 | 3.91 |

The XRPD overlay of the conversion showed that Form T might be an anhydrate/hydrate.

### EXAMPLE 21A: Preparation of Compound 1 Form U (Form U)

To the solution of Compound 1 (40 g) in DCM (240 mL) was slowly added n-heptane (140 mL) at 20-40° C. After stirred for 1 hour, another batch of n-heptane (20 mL) was added and then kept for 0.5 hours. Continually, n-heptane (20 mL) was added and kept for 0.5 hours, followed by addition of n-heptane (20 mL). Lastly, n-heptane (40 mL) was added and stirred for 12 hours at 20-40° C. The mixture was filtered, and the resulting cake was dried for 18 hours at 45-55° C. to give Compound 1 Form U (36.5 g), which could be used as a crystal seed.

To the solution of Compound 1 (6.6 kg) in DCM (51 kg) was added n-heptane (14 kg) at 25-35° C., and crystal seed (0.020 kg) was added. The mixture was stirred at 20-35° C. for about 4.5 hours, and four batches of n-heptane (2.0 kg+2.0 kg+4.0 kg+5.0 kg) were slowly added to the mixture, and then stirred at 20-35° C. for about 2 hours separately. The mixture was then stirred at 20-35° C. for about 16 hours. The mixture was filtered and washed with n-heptane (13 kg), and then the resulting cake was dried at 45-55° C. for 30 hours to give Compound 1 Form U as yellow solid (5.86 kg).

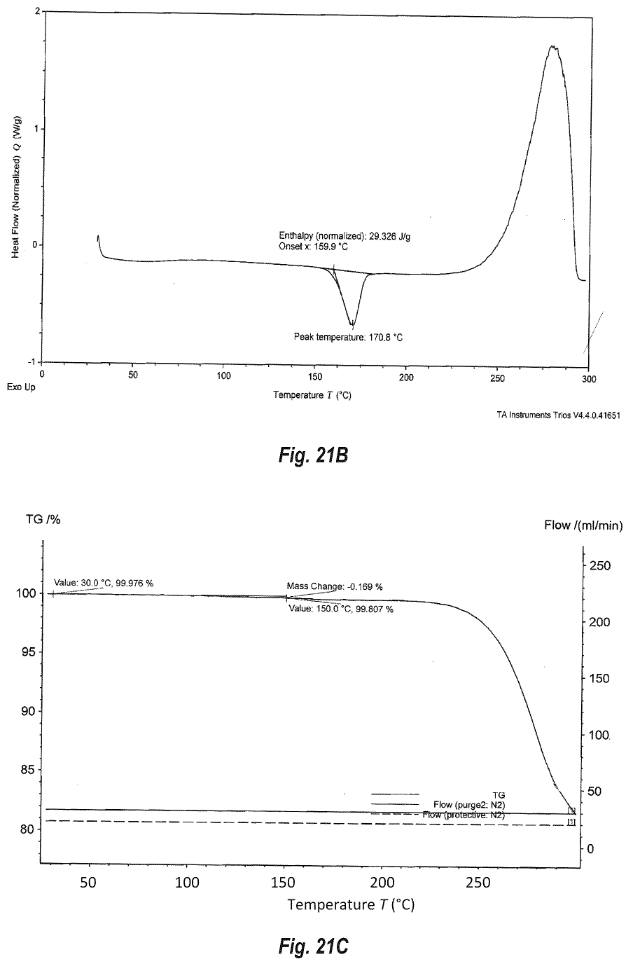

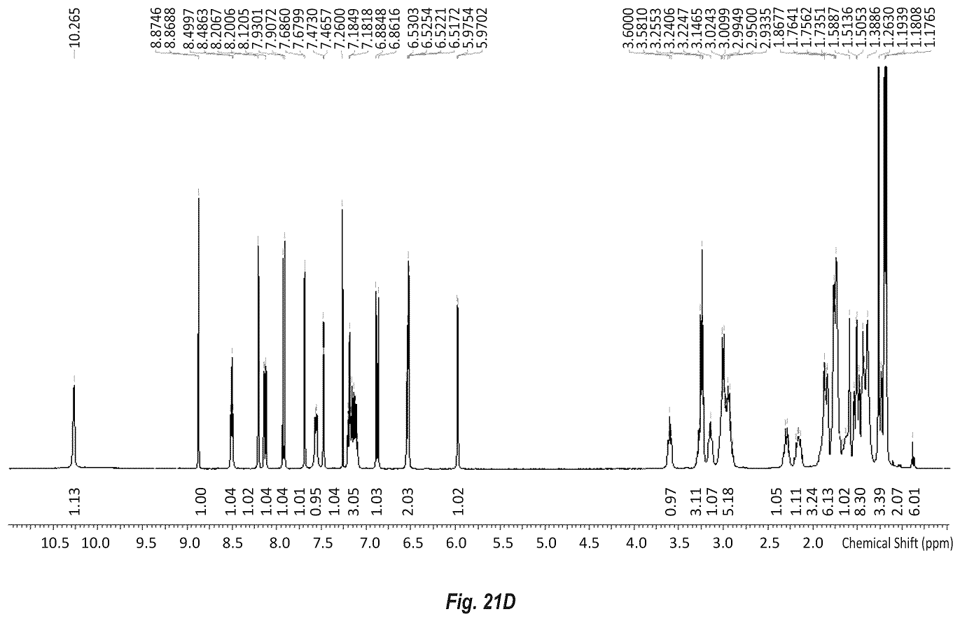

The X-ray powder diffraction (XRPD) pattern (conducted on Bruker D8 advanced X-Ray Powder diffractometer) was used to characterize the obtained Form U, which showed that Form U was in a crystalline form, see FIG. 21A. The characteristic peaks and percent peak intensities obtained from the XRPD analysis are listed in Table 21A.

TABLE 21A
XRPD pattern of Compound 1 Form U

| Pos. [°2θ] | d-spacing [Å] | Rel. Int. [%] |
|---|---|---|
| 6.968 | 12.67611 | 100.0 |
| 9.438 | 9.36287 | 13.1 |
| 10.237 | 8.63383 | 4.0 |
| 10.745 | 8.22714 | 4.0 |
| 11.282 | 7.83632 | 48.2 |
| 13.497 | 6.55533 | 53.3 |
| 13.920 | 6.35675 | 3.3 |
| 14.949 | 5.92133 | 8.4 |
| 15.019 | 5.89418 | 4.2 |
| 15.553 | 5.69298 | 32.7 |
| 16.073 | 5.50983 | 15.4 |
| 16.993 | 5.21352 | 16.6 |
| 17.116 | 5.17632 | 15.5 |
| 17.484 | 5.06834 | 13.1 |
| 18.036 | 4.91445 | 1.7 |
| 18.441 | 4.80742 | 1.7 |
| 18.908 | 4.68974 | 7.7 |
| 19.223 | 4.61343 | 2.2 |
| 19.549 | 4.53719 | 10.0 |
| 19.970 | 4.44261 | 13.9 |
| 20.515 | 4.32585 | 5.6 |
| 21.192 | 4.18916 | 13.9 |
| 21.613 | 4.10833 | 3.7 |
| 22.333 | 3.97756 | 2.0 |
| 22.600 | 3.93122 | 2.2 |
| 22.935 | 3.87453 | 2.0 |
| 23.633 | 3.76165 | 4.4 |
| 24.299 | 3.66000 | 9.0 |
| 25.736 | 3.45878 | 3.7 |
| 25.782 | 3.45273 | 4.4 |
| 26.147 | 3.40536 | 1.2 |
| 27.608 | 3.22838 | 2.6 |
| 28.462 | 3.13347 | 2.4 |
| 28.879 | 3.08910 | 1.0 |
| 29.317 | 3.04393 | 1.2 |

As TGA/DSC (conducted on NETZSCH TG 209 F1 Instrument, TA DSC 250) curves showed, a weight loss of 0.2% up to 150° C. and one endothermic peak at 170.8° C. (peak) was detected (FIG. 21B, and FIG. 21C). No signal of DCM was detected in ¹H NMR spectrum (FIG. 21D).

The DVS (Method B) cycle was conducted at 25° C., the sorption and desorption were revisable during the full DVS cycle, the water sorption is 1.4% at 95% RH humidity, the Compound 1 Form U has slightly hygroscopicity.

As the synthesis of Compound 1 showed in the international patent publication WO2019/210828, an acid intermediate (S)-2-((1H-pyrrolo[2,3-b]pyridin-5-yl)oxy)-4-(2-(2-(2-isopropylphenyl)pyrrolidin-1-yl)-7-azaspiro[3.5]nonan-7-yl)benzoic acid reacted with a sulfamide intermediate 4-((((1r,4r)-4-hydroxy-4-methylcyclohexyl)methyl)amino)-3-nitrobenzenesulfonamide to give Compound 1. At the same time, a dimer compound as a process impurity could be generated due to the azaindole part in compound 1 reacted with the acid intermediate. In the manufacture, only From U can unexpectedly remove the dimer impurity effectively. In one manufacture batch, the content of the dimer impurity in process control (IPC) was 0.4% (wt); after treating with EA crystallization, Form A was obtained and the content of the dimer impurity was still 0.4%; furtherly, the content of the dimer impurity was dropped to 0.22%, after treating with the recrystallization of THF/ACN mixture solution; finally, Form U was obtained after treating with the recrystallization of DCM/Heptane mixture solution, and the dimer impurity was not detected anymore.

### EXAMPLE 21B: Preparation of Compound 1 Form U

Amorphous Compound 1 (20 mg) was suspended in a mixture of DCM/n-heptane (1:1, v/v) at RT.

The suspension was subjected to slurry at RT by stirring for 1˜7 days, to obtain the Form U.

### EXAMPLE 21C: Preparation of Compound 1 Form U

Compound 1 (2.0 g) was dissolved into DCM (20 mL) at 40° C. The solution was added with heptane (15 mL) and stirred at 40° C., and then added with heptane (5.0 mL) at 40° C. The mixture was cooled down to RT and stirred, to generate precipitate. The precipitate was filtrated, washed with heptane and dried to give the product (1.4 g).

### EXAMPLE 22A: Compound 1 Amorphous Form (Amorphous)

Compound 1 (153.5 g) was dissolved in DCM (1.0 L) to give a clear solution. The solution was concentrated under reduced to remove solvent, and the residual was in a slurry with MTBE (1.0 L) and filtered. The filter cake was collected, dried under vacuum to give the product (137.5 g).

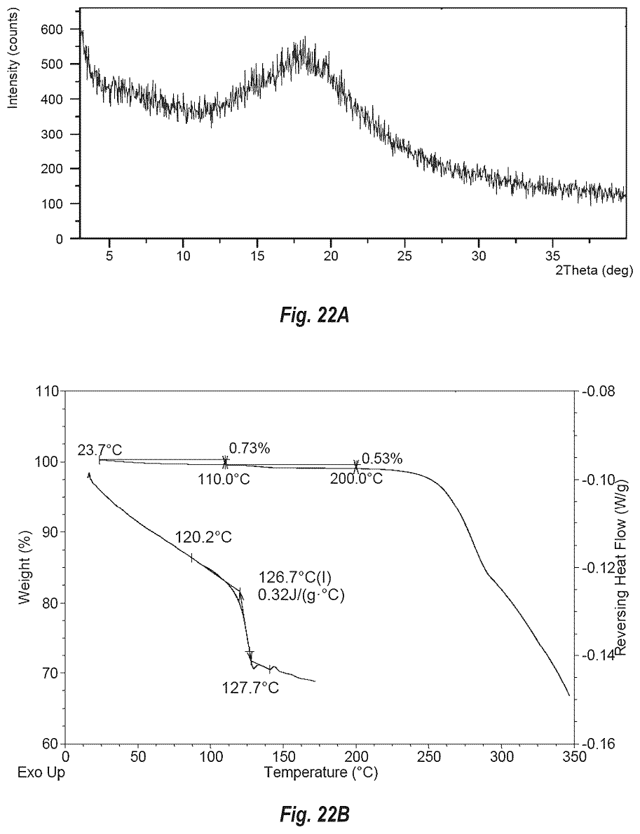

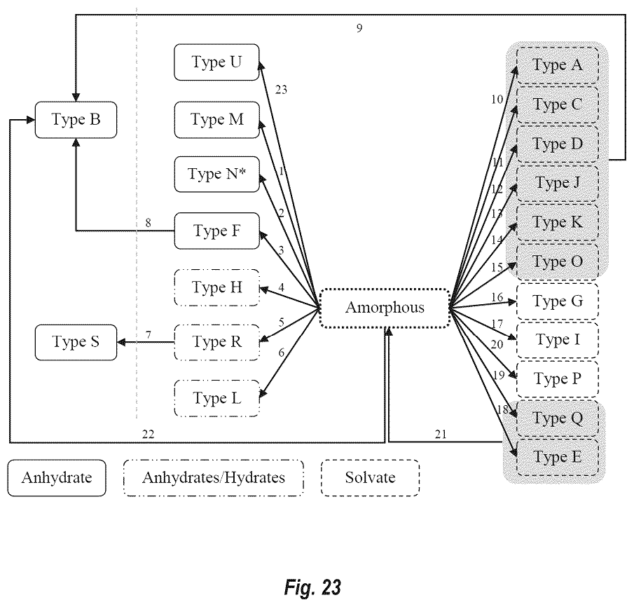

The obtained amorphous form showed an X-Ray Powder Diffraction (XRPD) pattern of FIG. 22A. TGA/DSC (FIG. 22B) results showed two stages of weight loss (0.7% up to 110° C., 0.5% from 110° C. to 200° C.) and a possible glass transition signal at 126.7° C. (middle) were observed. The chemical purity was determinated as 98.3% by high-performance liquid chromatography (HPLC). Results of DVS illustrated that with 1.8% water uptake at 80% RH/25° C.

### Physical Stability

To evaluate the physicochemical stability, store 1˜3 mg of Forms B, S, M, R, F, H and N samples were stored under 25° C./60% RH or 40° C./75% RH conditions for one week (unsealed) or 80° C. condition for 24 hrs (sealed).

XRPD overlays showed that no form change of Forms B, S, and M was observed.

After storing under 25° C./60% RH or 40° C./75% RH for one week, no form change was observed for Form R. After storing under 80° C./sealed for 24 hrs, Form R converted to a form which was similar to Form S.

For Form F, as XRPD overlay showed, a mixture of Forms F and B was observed after storing Form F sample under all the tested conditions.

XRPD patterns overlay displayed that no form change of Form H was observed under 25° C./60% RH or 40° C./75% RH for one week, but after placing under 80° C./sealed for 24 hrs, an obvious decrease of crystallinity was observed.

Form N converted to Form T, which could convert back to Form N when storing at RT for ˜3 days.

### Solid Form Solubility

The solubility of Compound 1 in different physical forms was tested in water, 0.1 N HCl, pH 4.5 acetate buffer and pH 6.8 phosphate buffer. At 24 hours of time point, the concentration of Compound 1 was detected by HPLC.

For Compound 1 amorphous, no Compound 1 was detected in water, pH 4.5 buffer and 6.8 buffer, but the corresponding solubility in 0.1 N HCl was 35.30 μg/mL. For Compound 1 Form A, the corresponding solubility in water, pH 4.5 and 6.8 buffer were 0.37 μg/mL, 0.76 μg/mL and 0.43 μg/mL, separately, but in 0.1 N HCl was 29.36 μg/mL. Thus, Form A showed higher solubility in water, pH 4.5 buffer and 6.8 buffer, but lower solubility in 0.1 N HCl, when compared with amorphous.

### Solid Form Stability

Evaluation of solid form stability of Form B was performed in acetone/H₂O system at 50° C. About 2 mg of Form B sample was used to slurry or shake in the acetone/H2O (1:9, v/v) and H2O solution (saturated by amorphous sample).

Crystalline status under slurring was tracked, and XRPD overlay showed that:
1) after slurring Form B in acetone/H2O (1:9, v/v) or H2O for about 4 days, a decrease of crystallinity was observed in Form B, including amorphous; and,
2) after slurrying Form B in H2O for about 4 hrs, no form change was observed.

In addition, crystalline status under shaking was also tracked. XRPD overlay illustrated that after shaking Form B in acetone/H₂O (1:9, v/v) or H₂O for about 4 hours or 4 days, no form change was observed, which implied that Form B might be influenced by mechanical force.

### Physical and Chemical Stability Test

Long term and accelerated stability studies were conducted in different physical forms of Compound 1, by storing the samples at 25±2° C./60±5% RH and 40±2° C./75±5% RH conditions for up to 6 months. And, the content of the total impurities of each sample was tested by HPLC.

For Compound 1 amorphous, the chemical purity of Compound 1 was reduced significantly, e.g., the total content of impurities increased from 2.10% to 4.2% when stored at 40±2° C./75±5% RH condition for 6 months, and many new impurities were tracked.

For Compound 1 Form A, the chemical purity of Compound 1 had no significant change, e.g., the total content of impurities only increased from 0.40% to 0.52% when stored at 40±2° C./75±5% RH condition for 6 months. In addition, no crystal form and optical purity changes were observed, but the content of solvent EA was reduced slightly, from about 9.5 to 8.8 (×10⁴ ppm).

For Compound 1 Form U, the chemical purity of Compound 1 had no significant change, e.g., the total content of impurities only increased from 0.40% to 0.72% when stored at 40±2° C./75±5% RH condition for 6 months. In addition, no crystal form and optical purity changes were observed.

Therefore, both Form A and Form U of Compound 1 showed better physical stability compared with its amorphous, and Form A showed better chemical stability.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

1-2. (canceled)

3. A crystalline form of 2-((1H-pyrrolo[2,3-b]pyridin-5-yl)oxy)-N-((4-((((1r,4r)-4-hydroxy-4-methylcyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)benzamide (Compound 1) is an EtOAc solvate, containing about 1 mol of EtOAc per mol, said form is designated as Form A.

4-16. (canceled)

17. A crystalline form of 2-((1H-pyrrolo[2,3-b]pyridin-5-yl)oxy)-N-((4-((((1r,4r)-4-hydroxy-4-methylcyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)benzamide (Compound 1) is an anhydrate, said form is designated as Form B.

18-27. (canceled)

28. An anhydrous crystalline form of 2-((1H-pyrrolo[2,3-b]pyridin-5-yl)oxy)-N-((4-((((1r,4r)-4-hydroxy-4-methylcyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)benzamide, wherein the crystalline form has an X-ray powder diffraction pattern comprising diffraction peaks having °2θ angle values at 11.3±0.1° and 24.3±0.1°.

29. (canceled)

30. The crystalline form according to claim 28, wherein the crystalline form has an X-ray powder diffraction pattern comprising diffraction peaks having °2θ angle values at 11.3±0.1°, 15.6±0.1° and 24.3±0.1°.

31. The crystalline form according to claim 28, wherein the crystalline form has an X-ray powder diffraction pattern comprising diffraction peaks having ° 2θ angle values at 11.3±0.1°, 15.6±0.1°, 21.2±0.1° and 24.3±0.1.

32. The crystalline form according to claim 28, wherein the crystalline form has an X-ray powder diffraction pattern comprising diffraction peaks having ° 2θ angle values at 11.3±0.1°, 13.5±0.1°, 15.6±0.1°, 21.2±0.1° and 24.3±0.1°.

33. The crystalline form according to claim 28, wherein the crystalline form has an X-ray powder diffraction pattern comprising diffraction peaks having ° 2θ angle values at 11.3±0.1°, 13.5±0.1°, 15.6±0.1°, 17.0±0.1°, 21.2±0.1° and 24.3±0.1°.

34. The crystalline form according to claim 28, wherein the crystalline form has an X-ray powder diffraction pattern comprising diffraction peaks having ° 2θ angle values at 11.3±0.1°, 13.5±0.1°, 15.6±0.1°, 17.0±0.1°, 19.5±0.1°, 21.2±0.1° and 24.3±0.1°.

35. The crystalline form according to claim 28, wherein the crystalline form has an X-ray powder diffraction pattern comprising diffraction peaks having ° 2θ angle values at 7.0±0.1°, 11.3±0.1°, 13.5±0.1°, 15.6±0.1°, 17.0±0.1°, 19.5±0.1°, 21.2±0.1° and 24.3±0.1°.

36. The crystalline form according to claim 28, wherein the crystalline form has an X-ray powder diffraction pattern comprising diffraction peaks having ° 2θ angle values at 7.0±0.1°, 11.3±0.1°, 13.5±0.1°, 15.6±0.1°, 17.0±0.1°, 19.5±0.1°, 20.0±0.1°, 21.2±0.1° and 24.3±0.1°.

37. The crystalline form according to claim 28, wherein the crystalline form has an X-ray powder diffraction pattern comprising diffraction peaks having ° 2θ angle values at 7.0±0.1°, 9.4±0.1, 11.3±0.1°, 13.5±0.1°, 15.6±0.1°, 17.0±0.1°, 19.5±0.1°, 20.0±0.1°, 21.2±0.1° and 24.3±0.1°.

38-39. (canceled)

40. The crystalline form according to claim 28, having an X-ray powder diffraction pattern substantially as shown in FIG. 21A.

41. The crystalline form according to claim 28, which has a differential scanning calorimetry (DSC) thermogram comprising one endotherm peak at about 171° C.

42. (canceled)

43. An amorphous form of 2-((1H-pyrrolo[2,3-b]pyridin-5-yl)oxy)-N-((4-((((1r,4r)-4-hydroxy-4-methylcyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)benzamide (Compound 1).

44-45. (canceled)

46. A pharmaceutical composition comprising the anhydrous crystalline form of claim 28 and one or more pharmaceutically acceptable excipients.

47. (canceled)

48. A method of treating a disease related to Bcl-2 proteins inhibition in a subject in need thereof comprising administering to the subject a therapeutically effective amount of the anhydrous crystalline form of claim 28.

49-52. (canceled)

53. The crystalline form according to claim 3, obtained by the process comprising any one of the following procedures:
a) dissolving Compound 1 in DCM, removing DCM, charging with EA, to obtain Form A;
b) dissolving Compound 1 in DCM, concentrating, charging with EA, exchanging DCM with EA, MeOH and EA separately, to obtain Form A;
c) dissolving Compound 1 in EA, heating and cooling, to obtain Form A; or
d) dissolving Compound 1 in THF/EtOAc (1:2, v/v) solvent mixture, evaporating, to obtain Form A.

54. The crystalline form according to any one claim 17, obtained by the process comprising any one of the following procedures:
a) dissolving Compound 1 in acetone, evaporating the solvent, to obtain Form B;
b) heating Form A, Form C, Form O to about 160° C. and cooling, to obtain Form B;
c) heating Form A stepwise isothermally to about 100° C., to obtain Form B;
d) heating Form D or Form J to about 130° C. and being isothermal, to obtain Form B; or
c) adding Form K into heptane, heating to about 100° C. and cooling, to obtain Form B.

55. The anhydrous crystalline form according to claim 28, obtained by the process comprising any one of the following procedures:
a) dissolving Compound 1 in DCM, adding n-heptane in batches and stirring, to obtain the anhydrous crystalline form; or
b) dissolving Compound 1 in the mixture of DCM/n-heptane (1:1, v/v) and stirring, to obtain the anhydrous crystalline form.

56. (canceled)

57. The amorphous form according to claim 43, obtained by the process comprising any one of the following procedures:
a) dissolving Compound 1 in DCM, drying, to obtain the amorphous form; or
b) dissolving Compound 1 in a mixture of solvent containing DCM, drying, to obtain the amorphous form.

58. (canceled)

59. A process for preparing a pharmaceutical composition comprising Compound 1, the process comprising mixing the solid form of Compound 1 according to claim 28 with at least one pharmaceutically acceptable excipient.
